(19) United States
(12) Patent Application Publication
(10) Pub. No.: US 2003/0104048 A1
(43) Pub. Date: Jun. 5, 2003

(54) Pharmaceutical dosage forms for highly hydrophilic materials

(75) Inventors: Mahesh V. Patel, Salt Lake City, UT (US); Feng-Jing Chen, Salt Lake City, UT (US); Steven L. Krill, Danbury, CT (US); Srinivasan Venkateshvaran, Salt Lake City, UT (US)

Correspondence Address: THORPE NORTH WESTERN, 8180 SOUTH 700 EAST, SUITE 200, P.O. BOX 1219, SANDY, UT, 84070, US

(73) Assignee: LIPOCINE, INC.

(21) Appl. No.: 10/158,206

(22) Filed: May 29, 2002

Related U.S. Application Data
(63) Continuation-in-part of application No. 09/898,553, filed on Jul. 2, 2001, now Pat. No. 6,451,339.
Application No. 09/898,553 is a continuation of application No. 09/258,654, filed on Feb. 26, 1999, now Pat. No. 6,294,192.
(63) Continuation-in-part of application No. 09/877,541, filed on Jun. 8, 2001.
Application No. 09/877,541 is a continuation-in-part of application No. 09/345,615, filed on Jun. 30, 1999, now Pat. No. 6,267,985.

(51) Int. Cl. A61K 9/00; A61K 9/48

(52) U.S. Cl. 424/451; 424/400

(57) ABSTRACT

Pharmaceutical dosage forms having a highly hydrophilic fill material and a shell encapsulating the fill material are disclosed and described. Generally, the shell has at least one plasticizing agent therein in order to provide the shell with an effective plasticity. In one aspect, the shell may have included therein an amount of plasticizing agent that is sufficient to provide the shell with an effective plasticity upon migration of a portion of the plasticizing agent into the fill material. In another aspect, the plasticizing agent may have a solubility in the fill material of less than about 10% w/w. In yet another aspect, a combination of a plasticizing agent, and a plasticizing agent having a solubility in the fill material of less than about 10% w/w, may be presented in a total amount sufficient to provide the shell with an effective plasticity upon migration of plasticizing agent into the fill material.

### PRIORITY DATA

This application is a continuation-in-part of U.S. patent application Ser. No. 09/898,553, filed on Jul. 2, 2001, which is a continuation of U.S. patent application Ser. No. 09/258,654, filed Feb. 26, 1999, now issued as U.S. Pat. No. 6,294,192. This application is also a continuation-in-part of U.S. patent application Ser. No. 09/877,541, filed on Jun. 8, 2001, which is a continuation of U.S. patent application Ser. No. 09/345,615, filed on Jun. 30, 1999, now issued as U.S. Pat. No. 6,267,985. Each of the above-recited patents and patent applications, as well as each of the additional references set forth in this patent application are incorporated herein by reference.

### FIELD OF THE INVENTION

The present invention relates to pharmaceutical dosage forms that include a highly hydrophilic fill material and shell that encapsulates the fill material. Accordingly, the present invention involves the fields of chemistry, pharmaceutical sciences, and medicine.

### BACKGROUND

Oral capsules are a well-known dosage form for administering various agents into the body through the gastrointestinal tract. Generally speaking, such capsules have two basic components, namely, a fill material that includes a pharmaceutically active agent, and a shell that encapsulates the fill material. Upon administration, the fill material is released and absorbed by the body as the shell degrades under various digestive forces.

Many specific constituents have been used to form the shell of various capsule formulations. One basic component is a matrix, or film forming material, such as gelatin, hydroxypropyl methyl cellulose (HPMC), gums, or other polymeric materials. Other, additives are often included in the shell to control the physical characteristics thereof. One such additive is a plasticizing agent that is used to control the softness or pliability of the shell. Regardless of whether the shell is prepared to be a hard or soft shell, certain amounts of plasticizing agents are important in order to keep the shell from becoming overly brittle.

Since its first inception, the concept of a capsule dosage form has evolved to include a variety of specific formulations which attain certain desired physical and performance properties. For example, in addition to a solid fill material, liquid or semi-solid fills have been employed in order to enable a more rapid release and an increased absorption of the pharmaceutically active agent. Further, both hard and soft shells have been used in order to vary the release timing of the pharmaceutically active agent. In short, by the variation of the shell and fill material constituents, improved capsule dosage forms have been produced.

One common constituent of the fill material is a carrier, or vehicle in which the pharmaceutically active agent to be delivered is dissolved or dispersed. Traditionally, fat soluble vitamins, such as vitamin E and digestible oils, such as triglycrides and fatty acids have been employed as the major constituent either as the active ingredient itself or as the carrier or vehicle for dissolving or dispersing the active ingredient. In general, these non-hygroscopic and non-glycerol solublizing materials enjoy good compatibility with traditional gelatin capsule shells that utilize glycerol as the plasticizer. However, the performance of these dosage forms frequently suffer from inconsistent and poor absorption of the active ingredients due to the lack of water dispersibility of such fill materials in vivo.

As a result, many types of carriers with improved water dispersibility have been sought and used. One class of vehicle that has been used in the fill is liquid polyethylene glycols (PEG) with a molecular weight of 100-600. However, since PEG is not a surfactant, it provides insufficient solubilization for a wide range of active ingredients once administered to the GI tract. Further, these materials suffer from the disadvantage of making the capsules brittle upon storage because their hygroscopic nature tend to draw water and other constituents, such as plasticizers out of the shell over time, as reported in U.S. Pat. Nos. 4,744,988 and 4,780,316.

Excessive brittleness interferes with the functionality of capsule dosage forms in a number of ways. First, an excessively brittle capsule may actually crack or burst prior to administration, thus allowing the fill material to leak therefrom. Further, a capsule that is too brittle may take too long to dissolve in gastric juices, and therefore the encapsulated active ingredient may not be released and absorbed as it intended to be. These and other issues caused by capsule embrittlement most often render the dosage form useless and a embrittlement inhibiting composition is required to impart physical stability and durability to the capsule.

Another problem that has been recognized with many fill materials, such as 1,2-propylene glycols), is their propensity to migrate into the shell, and thus overly soften it. One example of this phenomenon is contained in U.S. Pat. No. 5,985,321. Overly softened shells experience a several performance disadvantages, and further, the loss of propylene glycol from the fill material may upset an established balance of constituents that is required for sufficient drug loading capacity of the formulation and proper delivery and absorption of the active ingredient in the gastrointestinal tract. Therefore, propylene glycol was added to the shell as well to counteract the migration of it from the fill.

As a result, capsule dosage forms that include a fill material containing constituents capable of holding and delivering a wide variety of drugs, such as hygroscopic and hydrophilic carriers, that limit the movement of constituents from the shell into the fill material, and from the fill material into the shell, thus maintaining desired shell integrity and performance, continue to be sought through ongoing research and development efforts.

### SUMMARY OF THE INVENTION

Accordingly, the present invention encompasses pharmaceutical dosage forms having a highly hydrophilic fill material that is encapsulated by a shell which maintains an effective plasticity despite the hydrophilicity of the fill material.

In one aspect, the dosage form may include a fill material may having a carrier of at least about 40% w/w of a hydrophilic surfactant, and at least one pharmaceutically active agent, and a shell encapsulating the fill material which contains at least one plasticizing agent in an amount sufficient to maintain an effective shell plasticity upon migration of a portion of the plasticizing agent into the fill material.

In another aspect, the dosage form may include a fill material having a carrier of at least about 40% w/w of a hydrophilic surfactant, and at least one pharmaceutically active agent, and a shell encapsulating the fill material which contains an effective amount of a plasticizing agent having a solubility of less than 10% w/w in the fill material.

In yet another aspect of the invention, the dosage form may include a fill material having a carrier of at least about 40% w/w of a hydrophilic surfactant, and at least one pharmaceutically active agent, and a shell encapsulating the fill material, said shell containing a first plasticizing and a second plasticizing agent, said second plasticizing agent having a solubility in the fill material of less than about 10% w/w agent, wherein the first and second plasticizing agents are present in amounts sufficient to maintain an effective shell plasticity upon migration of a portion of either plasticizing agent into the fill material.

It is also an aspect of the present invention that the plasticizing agent(s) is present in an amount that the disintegration of the dosage form and/or the release of the fill material is not significantly alterted (becomes slower or incomplete) after storage.

It is another aspect of the present invention that the plasticizing agent(s) is present in an amount that the disintegration of the dosage form and/or the release of the fill material is not significantly alterted (becomes slower or incomplete) after storage, even if there is any chemical degradation or denaturation occurring in the shell, such as crosslinking of gelatin capsules by aldehyde substances.

There has thus been outlined, rather broadly, various features of the present invention so that the detailed description thereof that follows may be better understood, and so that the present contribution to the art may be better appreciated. Other features of the present invention will become clearer from the following detailed description of the invention, taken with the accompanying claims, or may be learned by the practice of the invention.

### BRIEF DESCRIPTION OF THE DRAWINGS

FIG. 1 is a graphical representation of release rate testing results achieved by oral dosage forms containing a highly hydrophilic fill material as used in the present invention, and traditional shell compositions used for moderately to low hydrophilic materials, such as PEG, following storage under varying conditions for a period of 4 weeks, as compared to freshly made conventional oral dosage forms.

### DETAILED DESCRIPTION OF THE INVENTION

Before the present pharmaceutical dosage forms are disclosed and described, it is to be understood that the present invention is not limited to the particular process steps and materials disclosed herein, but is extended to equivalents thereof as would be recognized by those ordinarily skilled in the relevant arts. It should also be understood that terminology employed herein is used for the purpose of describing particular embodiments only and is not intended to be limiting.

Definitions

In describing and claiming the present invention, the following terminology will be used.

The singular forms “a,” “an,” and, “the” include plural referents unless the context clearly dictates otherwise. Thus, for example, reference to a fill material containing “a hydrophilic carrier” includes one or more hydrophilic carriers, reference to “an additive” includes reference to one or more of such additives, and reference to “the plasticizing agent” includes reference to one or more of such agents.

The terms “composition” and “formulation may be used interchangeably herein.

As used herein, “matrix forming material” and “film forming material may be used interchangeably, and refer to materials that are known to those of ordinary skill in the art as suitable for use in forming a shell of a typical capsule dosage form. Examples of such materials include without limitation, various gelatins, hydroxypropyl methyl cellulose (HPMC), starches, polymers, and gum acacia.

As used herein “shell” refers to a barrier that encapsulates, surrounds, or encompasses at least a portion of a material or an object. In the pharmaceutical arts, capsule dosage forms are well known to include a shell as an essential component that surrounds a fill material. A variety of specific materials and methods for the formation of such shells, are well known to those of ordinary skill in the art.

As used herein, an “effective amount,” and “sufficient amount” may be used interchangeably, and refer to an amount of a substance that is sufficient to achieve an intended purpose or objective. For example, a sufficient, or effective amount of a suspending agent would be the minimum amount of agent required to effectively suspend one substance, such as a pharmaceutically active agent, in a carrier. The determination of an effective amount is well within the ordinary skill in the art of pharmaceutical, neutraceutical, herbaceutical, cosmetic, and medical sciences. See, for example, Meiner and Tonascia, “Clinical Trials: Design, Conduct, and Analysis,” Monographs in Epidemiology and Biostatistics, Vol. 8 (1986).

As used herein, “pharmaceutically active agent,” “bioactive agent,” “therapeutic agent,” “active agent,” and “drug” may be used interchangeably herein, and refer to a substance, such as a chemical compound or complex, that has a measurable beneficial physiological effect on the body, such as a therapeutic effect in treatment of a disease or disorder, when administered in an effective amount. Further, when these terms are used, or when a particular active agent is specifically identified by name or category, it is to be understood that such recitation is intended to include the active agent per se, as well as pharmaceutically acceptable, pharmacologically active derivatives thereof, or compounds significantly related thereto, including without limitation, salts, esters, amides, prodrugs, active metabolites, isomers, fragments, analogs, etc.

Concentrations, amounts, solubilities, particle size, wavelength, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited.

As an illustration, a concentration range of “about 4% w/w to about 60% w/w” should be interpreted to include not only the explicitly recited concentration of about 4% w/w to about 60% w/w, but also include individual concentrations and the sub-ranges within the indicated range. Thus, included in this numerical range are individual concentrations such as 4% w/w, 10% w/w, 23% w/w, and 46% w/w, and sub-ranges such as from 10% w/w to 50% w/w, from 20% w/w to 40% w/w, from 25% w/w to 35% w/w, from 15% w/w to 20% w/w, etc.

This same principle applies to ranges reciting only one numerical value. Furthermore, such an interpretation should apply regardless of the breadth of the range or the characteristics being described.

Invention

Applicants have discovered pharmaceutical dosage forms having a shell that retains an effective plasticity while encapsulating a highly hydrophilic fill material. Such dosage forms present a number of advantages including increased freedom in formulating, processing and manufacturing specific dosage forms, increased absorption and/or efficacy of the active ingredient, more consistent performance of the dosage form with respect to disintegration of the dosage and dissolution/ solubilization of the active ingredient and improved storage stability.

### A. Shell Formulations

The shell of the present invention may be either a hard or soft capsule shell, and includes a number of fundamental constituents as will be recognized by those of ordinary skill in the art, namely a matrix forming material, and at least one plasticizing agent. A wide variety of matrix forming materials are suitable for use in the dosage forms of the present invention, and the selection of specific materials may be based, at least in part, on factors such as the specific results to be achieved. Examples of specific materials include without limitation, gelatins, including type A gelatins, such as the gelatin derived from acid-treated pigskins, and type B gelatins, such as those derived from alkali-treated bovine bones and hides, hydroxypropyl methylcellulose (HPMC), starches, and gum acacia. Other specific matrix forming materials that may be particularly desired in view of a given overall dosage form can be determined by those of ordinary skill in the art.

The specific amount of matrix forming material used in the shell formulation may be determined in part by a variety of factors, including the type of shell to be formed (i.e. hard or soft), and by the amount and type of other constituents or additives that are to be included in the shell. However, in one aspect, the amount of matrix forming material may be from about 20% w/w to about 70% w/w of the shell. In another aspect, the amount may be from about 30% w/w to about 50% w/w of the shell.

Many plasticizing agents are known, and may also be used in the shell of the present dosage form. One basis for selecting a particular plasticizing agent may be the solubility of that agent in a specific hydrophilic fill material to be used. In one aspect, the plasticizing agent may have a solubility of less than about 10% w/w in the fill material. In another aspect, the solubility of the plasticizing agent in the fill material may be less than about 5% w/w. In yet another aspect, the solubility may be less than about 1% w/w. In a further aspect, the solubility of the plasticizing agent may be less than about 0.5% w/w. Lowered solubility in the specific hydrophilic fill material substantially impedes the migration of the plasticizing agent out of the shell and into the fill material. Examples of specific plasticizing agents displaying such limited solubilities in many hydrophilic surfactant materials include without limitation: sorbitol, sorbitanes, xylitol, maltitol, maltitol syrup, partially dehydrated hydrogenated glucose syrups, hydrogenated starch hydrolysate, polyhydric alcohols having an equilibrium relative humidity of greater than or equal to 80%, carrageenan, polyglycerol, non-crystallizing solutions of sorbitol, glucose, fructose, glucose syrups, and mixtures and equivalents thereof.

Whether the plasticizing agent selected and used is one that has a low solubility in the fill material or not, in accordance with one aspect of the invention, the plasticizing agent may be presented in an amount that is sufficient to maintain an effective shell plasticity upon migration of a portion of the plasticizing agent from the shell and into the fill and/or may be present in a sufficient amount to maintain a desirable dissolution/disintegration profile with respect to the rate and the extent release and/or dispersing of the encapsulated active agent in a specific dissolution medium or upon administration inside the GI tract. The exact amount of plasticizing agent required to compensate for the plasticizing agent anticipated to be lost may depend on a variety of factors, such as the specific fill material and solubility of the plasticizing agent therein. However, those of ordinary skill in the art will be able to readily determine approximate amounts required to maintain effective shell plasticity based on the known characteristics presented by a given dosage form, and will further be able to identify specific amounts through routine experimentation with the dosage form. In one aspect of the invention, such an amount of plasticizing agent may be from about 4% w/w to about 60% w/w of the shell. In another aspect, the amount may be from about 10% w/w to about 35% w/w.

An additional option for maintaining effective shell plasticity and/or a desirable dissolution/disintegration profile of the encapsulated active agent in view of the highly hydrophilic fill material is to include a combination of plasticizing agents in the shell in a total amount sufficient to maintain effective shell plasticity upon migration of a portion of either or both agents into the fill material. In one aspect of the invention, such a combination may include a first plasticizing agent, and a second plasticizing agent having a limited solubility in the fill material as recited above. The total amounts and ratios of each ingredient required to maintain an effective plasticity may be determined by one of ordinary skill in the art in the manners already indicated. While a variety of ratios and amounts are contemplated, in one aspect, the total amount of combined plasticizing agent may be within the ranges already established for plasticizing agents herein.

In addition to the components of a matrix forming material and the at least one plasticizing agent, the shells used in the dosage forms of the present invention may include additional additives as required, in order to achieve a specifically desired formulation or result. Examples of such additives may include without limitation, coloring agents, antioxidants, preservatives, surfactants, and mixtures thereof. Specific amounts of these additives, as well as others not specifically recited will be readily determined by those of ordinary skill in the art, consistent with a working knowledge thereof, and the principles set forth herein.

In addition to the above recited devices and methods for maintaining the flexibility, or plasticity of a shell encapsulating a highly hydrophilic material, another approach encompassed by the present invention, is the use of a hydrophobic coating on a surface of the shell. Specifically, it is thought that by placing a hydrophobic coating along an inner surface of the shell, that water and plasticizer may be effectively stopped from migrating into the the fill material, or at least that such migration may be slowed. Further, when such a coating is provided along an outer surface of the shell it is thought that the coating prevents the absorption of moisture from the outside environment, and its resultant migration into the fill material, or that at least, such is slowed. In addition to slowing or preventing the migration of water and plasticizers into the fill material, use of such coatings is thought to prevent or slow the migration of plasticizers from the shell and into the fill material. Such migration is known to cause over-softening or “sweating” of the shell, which can be can be as detrimental to the performance of the dosage form as embrittling of the shell.

Either coating may be used separately in various embodiments of the present invention, or a combination of coatings may be used. Such coatings may further be employed with virtually any specific dosage form or shell formulation as contemplated herein. Further, a variety of hydrophobic, or water impermeable materials may be used for the coating as will be recognized by those of ordinary skill in the art, such as oils, petroleum waxes, etc.

### B. Fill Material

The fill materials of the present oral dosage forms contain at least one pharmaceutically active agent, or drug, and a carrier, or vehicle, in which the drug is dissolved or dispersed. As a general matter, the carrier typically includes a hydrophilic surfactant in a significantly higher amount than found in a typical emulsion pre-concentrate or a typical microemulsion pre-concentrate. It is thought that such amounts present a number of performance and efficacy advantages, including without limitation increased solubility of the active agent in the fill material, increased dispersibility of the fill material in the gastrointestinal tract. Thus, larger doses of a therapeutic agent can be consistantly delivered and absorbed with greater speed and efficiency.

In general, the fill material of the present invention includes at least about 40% w/w of a hydrophilic surfactant. However, in one aspect, the hydrophilic surfactant may comprise at least about 50% of the carrier. In yet another aspect, the hydrophilic surfactant may comprise at least about 60% w/w of the carrier. Furthermore, a lipophilic additive, such as a lipophilic surfactant or a triglyceride may be included in the fill material. Other additives may also be included, such as antioxidants, bufferants, antifoaming agents, detackifiers, preservatives, chelating agents, viscomodulators, tonicifiers, flavorants, colorants, odorants, opacifiers, stabilizing agents, solubilizers, binders, fillers, plasticizing agents, lubricants, and mixtures thereof. The specific type and amount of additive may be selected by one of ordinary skill in the art, in order to provide a dosage form with particular characteristics.

1. Triglycerides

One specific lipohilic additive that may be combined with the hydrophilic surfactant carrier of the present fill material is a triglyceride. Examples of suitable triglycerides are shown in Table 1. In general, these triglycerides are readily available from commercial sources. For several triglycerides, representative commercial products and/or commercial suppliers are listed.

TABLE 1
Triglycerides

| Triglyceride | Commercial Source |
|---|---|
| Aceituno oil | |
| Almond oil | Super Refined Almond Oil(Croda) |
| Araehis oil | |
| Babassu oil | |
| Blackcurrant seed oil | |
| Borage oil | |
| Buffalo ground oil | |
| Candlenut oil | |
| Canola oil | Lipex 108 (Abitec) |
| Caster oil | |
| Chinese vegetable tallow oil | |
| Cocoa butter | |
| Coconut oil | Pureco 76 (Abitec) |
| Coffee seed oil | |
| Corn oil | Super Refined Corn Oil (Croda) |
| Cottonseed oil | Super Refined Cottonseed Oil(Croda) |
| Crambe oil | |
| Cuphea species oil | |
| Evening primrose oil | |
| Grapeseed oil | |
| Groundnut oil | |
| Hemp seed oil | |
| Illipe butter | |
| Kapok seed oil | |
| Linseed oil | |
| Menhaden oil | Super Refined Menhaden Oil (Croda) |
| Mowrah butter | |
| Mustard seed oil | |
| Oiticica oil | |
| Olive oil | Super Refined Olive Oil (Croda) |
| Palm oil | |
| Palm kernel oil | |
| Peanut oil | Super Refined Peanut Oil(Croda) |
| Poppy seed oil | |
| Rapeseed oil | |
| Rice bran oil | |
| Safflower oil | Super Refined Safflower Oil(Croda) |
| Sal fat | |
| Sesame oil | Super Refined Sesame Oil(Croda) |
| Shark liver oil | Super Refined Shark Liver Oil(Croda) |
| Shea nut oil | |
| Soybean oil | Super Refined Soybean Oil(Croda) |
| Stillingia oil | |
| Sunflower oil | |
| Tall oil | |
| Tea sead oil | |
| Tobacco seed oil | |
| Tung oil (China wood oil) | |
| Ucuhuba | |
| Vernonia oil | |
| Wheat germ oil | Super Refined Wheat Germ Oil(Croda) |
| Hydrogenated caster oil | Castorwax |
| Hydrogenated coconut oil | Pureco 100 (Abitec) |
| Hydrogenated cottonseed oil | Dritex C (Abitec) |
| Hydrogenated palm oil | Dritex PST (Abitec); Softisan 154 (Huls) |
| Hydrogenated soybean oil | Sterotex HM NF (Abitec); Dritex S (Abitec) |
| Hydrogenated vegetable oil | Sterotex NF (Abitec): Hydrokote M (Abitec) |
| Hydrogenated cottonseed and caster oil | Sterotex K (Abitec) |
| Partially hydrogenated soybean oil | Hydrokote AP5 (Abitec) |
| Partially soy and cottonseed oil | Apex B (Abitec) |
| Glyceryl tributyrate | (Sigma) |
| Glyceryl tricaproate | (Sigma) |
| Glyceryl tricaprylate | (Sigma) |
| Glyceryl tricaprate | Captex 1000 (Abitec) |
| Glyceryl trundecanoate | Captex 8227 (Abitec) |
| Glyceryl trilaurate | (Sigma) |
| Glyceryl trimyristate | Dynasan 114 (Huls) |
| Glyceryl tripalmitate | Dynasan 116 (Huls) |
| Glyceryl tristearate | Dynasan 118 (Huls) |
| Glyceryl triarcidate | (Sigma) |
| Glyceryl trimyristoleate | (Sigma) |
| Glyceryl tripalmitoleate | (Sigma) |
| Glyceryl trioleate | (Sigma) |
| Glyceryl trilinoleate | (Sigma) |
| Glyceryl trilinolenate | (Sigma) |
| Glyceryl tricaprylate/caprate | Captex 300 (Abitec); Captex 355 |
| | (Abitec); Miglyol 810 (Huls); |
| | Miglyol 812 (Huls) |
| Glyceryl tricaprylate/caprate/laurate | Captex 350 (Abitec) |
| Glyceryl tricaprylate/caprate/linoleate | Captex 810 (Abitec); Miglyol |
| | 818 (Huls) |
| Glyceryl tricaprylate/caprate/stearate | Softisan 378 (Huls); (Larodan) |
| Glyceryl tricaprylate/laurate/stearate | (Larodan) |
| Glyceryl 1,2-caprylate-3-linoleate | (Larodan) |
| Glyceryl 1,2-caprate-3-stearate | (Larodan) |
| Glyceryl 1,2-laurate-3-myristate | (Larodan) |
| Glyceryl 1,2-myristate-3-laurate | (Larodan) |
| Glyceryl 1,3-palmitate-2-butyrate | (Larodan) |
| Glyceryl 1,3-stearate-2-caprate | (Larodan) |
| Glyceryl 1,2-linoleate-3-caprylate | (Larodan) |

Fractionated triglycerides, modified triglycerides, synthetic triglycerides, and mixtures of triglycerides are also within the scope of the invention.

Preferred triglycerides include vegetable oils, fish oils, animal fats, hydrogenated vegetable oils, partially hydrogenated vegetable oils, medium and long-chain triglycerides, and structured triglycerides. It should be appreciated that several commercial surfactant compositions contain small to moderate amounts of triglycerides, typically as a result of incomplete reaction of a triglyceride starting material in, for example, a transesterification reaction. Such commercial surfactant compositions, while nominally referred to as “surfactants”, may be suitable to provide a desired triglyceride amount. Examples of commercial surfactant compositions containing triglycerides include some members of the surfactant families Gelucires (Gattefosse), Maisines (Gattefosse), and Imwitors (Huls). Specific examples of these compositions are:

Gelucire 44/14 (saturated polyglycolized glycerides)

Gelucire 50/13 (saturated polyglycolized glycerides)

Gelucire 53/10 (saturated polyglycolized glycerides)

Gelucire 33/01 (semi-synthetic triglycerides of C<8>-C<18> saturated fatty acids)

Gelucire 39/01 (semi-synthetic glycerides)

other Gelucires, such as 37/06, 43/01, 35/10, 37/02, 46/07, 48/09, 50/02, 62/05, etc.

Maisine 35-I (linoleic glycerides)

Imwitor 742 (caprylic/capric glycerides)

Still other commercial surfactant compositions having significant triglyceride content are known to those skilled in the art. It should be appreciated that such compositions, which contain triglycerides as well as surfactants, may be suitable to provide a triglyceride constituent for the purposes of the present invention.

Among the above-listed triglycerides, preferred triglycerides include: almond oil; babassu oil; borage oil; blackcurrant seed oil; canola oil; castor oil; coconut oil; corn oil; cottonseed oil; evening primrose oil; grapeseed oil; groundnut oil; mustard seed oil; olive oil; palm oil; palm kernel oil; peanut oil; rapeseed oil; safflower oil; sesame oil; shark liver oil; soybean oil; sunflower oil; hydrogenated castor oil; hydrogenated coconut oil; hydrogenated palm oil; hydrogenated soybean oil; hydrogenated vegetable oil; hydrogenated cottonseed and castor oil; partially hydrogenated soybean oil; partially soy and cottonseed oil; glyceryl tricaproate; glyceryl tricaprylate; glyceryl tricaprate; glyceryl triundecanoate; glyceryl trilaurate; glyceryl trioleate; glyceryl trilinoleate; glyceryl trilinolenate; glyceryl tricaprylate/caprate; glyceryl tricaprylate/caprate/laurate; glyceryl tricaprylate/caprate/linoleate; and glyceryl tricaprylate/caprate/stearate. Other preferred triglycerides are saturated polyglycolized glycerides (Gelucire 44/14, Gelucire 50/13 and Gelucire 53/10), linoleic glycerides (Maisine 35-I), and caprylic/capric glycerides (Imwitor 742).

Among the preferred triglycerides, more preferred triglycerides include: coconut oil; corn oil; olive oil; palm oil; peanut oil; safflower oil; sesame oil; soybean oil; hydrogenated castor oil; hydrogenated coconut oil; partially hydrogenated soybean oil; glyceryl tricaprate; glyceryl trilaurate; glyceryl trioleate; glyceryl trilinoleate; glyceryl tricaprylate/caprate; glyceryl tricaprylate/caprate/laurate; glyceryl tricaprylate/caprate/linoleate; glyceryl tricaprylate/caprate/stearate; saturated polyglycolized glycerides (Gelucire 44/14, Gelucire 14 50/13 and Gelucire 53/10); linoleic glycerides (Maisine 35-I); and caprylic/capric glycerides (Imwitor 742).

2. Surfactants

As is well known in the art, the terms “hydrophilic” and “lipophilic” are relative terms. To function as a surfactant, a compound must necessarily include polar or charged hydrophilic moieties as well as non-polar lipophilic (hydrophobic) moieties. In other words, a surfactant compound must be amphiphilic. An empirical parameter commonly used to characterize the relative hydrophilicity and lipophilicity of non-ionic amphiphilic compounds is the hydrophilic-lipophilic balance (“HLB” value). Surfactants with lower HLB values are more lipophilic, and have greater solubility in oils, while surfactants with higher HLB values are more hydrophilic, and have greater solubility in aqueous solutions.

Using HLB values as a rough guide, hydrophilic surfactants are generally considered to be those compounds having an HLB value of greater than about 10, as well as anionic, cationic, or zwitterionic compounds for which the HLB scale is not generally applicable. Similarly, lipophilic surfactants are compounds having an HLB value of less than about 10.

It should be appreciated that the HLB value of a surfactant is merely a rough guide generally used to enable formulation of industrial, pharmaceutical and cosmetic emulsions. For many important surfactants, including several polyethoxylated surfactants, it has been reported that HLB values can differ by as much as about 8 HLB units, depending upon the empirical method chosen to determine the HLB value (Schott, J Pharm. Sciences, 79(1), 87-88 (1990)). Likewise, for certain polypropylene oxide containing block copolymers (PLURONIC® surfactants, BASF Corp.), the HLB values may not accurately reflect the true physical chemical nature of the compounds. Finally, commercial surfactant products are generally not pure compounds, but are complex mixtures of compounds, and the HLB value reported for a particular compound may more accurately be characteristic of the commercial product of which the compound is a major component. Different commercial products having the same primary surfactant component can, and typically do, have different HLB values. In addition, a certain amount of lot-to-lot variability is expected even for a single commercial surfactant product. Keeping these inherent difficulties in mind, and using HLB values as a guide, one skilled in the art can readily identify surfactants having suitable hydrophilicity or lipophilicity for use in the present invention, as described herein.

The hydrophilic surfactant can be any hydrophilic surfactant suitable for use in pharmaceutical compositions. Such surfactants can be anionic, cationic, zwitterionic or non-ionic, although non-ionic hydrophilic surfactants are presently preferred. As discussed above, these non-ionic hydrophilic surfactants will generally have HLB values greater than about 10. Mixtures of hydrophilic surfactants are also within the scope of the invention.

Similarly, the lipophilic surfactant can be any lipophilic surfactant suitable for use in pharmaceutical compositions. In general, suitable lipophilic surfactants will have an HLB value less than about 10. Mixtures of lipophilic surfactants are also within the scope of the invention.

The choice of specific lipophilic and hydrophilic surfactants should be made keeping in mind the particular therapeutic agent to be used in the composition, and the range of polarity appropriate for the chosen therapeutic agent, as discussed in more detail below. With these general principles in mind, a very broad range of surfactants is suitable for use in the present invention. Such surfactants can be grouped into the following general chemical classes detailed in the Tables below. The HLB values given in the Tables below generally represent the HLB value as reported by the manufacturer of the corresponding commercial product. In cases where more than one commercial product is listed, the HLB value in the Tables is the value as reported for one of the commercial products, a rough average of the reported values, or a value that, in the judgment of the Applicants, is more reliable. It should be emphasized that the invention is not limited to the surfactants in the following Tables, which show representative, but not exclusive, lists of available surfactants.

2.1. Polyethoxylated Fatty Acids

Although polyethylene glycol (PEG) itself does not function as a surfactant, a variety of PEG-fatty acid esters do. Among the PEG-fatty acid monoesters, esters of lauric acid, oleic acid, and stearic acid are most useful. Among the surfactants of Table 1, preferred hydrophilic surfactants include PEG-8 laurate, PEG-8 oleate, PEG-8 stearate, PEG-9 oleate, PEG-10 laurate, PEG-10 oleate, PEG-12 laurate, PEG-12 oleate, PEG-15 oleate, PEG-20 laurate and PEG-20 oleate. Examples of polyethoxylated fatty acid monoester surfactants commercially available are shown in Table 2.

TABLE 2
PEG-Fatty Acid Monoester Surfactants

| COMPOUND | COMMERCIAL PRODUCT (Supplier) | HLB |
|---|---|---|
| PEG 4-100 monolaurate | Crodet L series (Croda) | >9 |
| PEG 4-100 monooleate | Crodet O series (Croda) | >8 |
| PEG 4-100 monostearate | Crodet S series (Croda), Myrj Series | >6 |
| | (Atlas/ICI) | |
| PEG 400 distearate | Cithrol 4DS series (Croda) | >10 |
| PEG 100, 200, 300 monolaurate | Cithrol ML series (Croda) | >10 |
| PEG 100, 200, 300 monooleate | Cithrol MO series (Croda) | >10 |
| PEG 400 dioleate | Cithrol 4DO series (Croda) | >10 |
| PEG 400-1000 monostearate | Cithrol MS series (Croda) | >10 |
| PEG-1 stearate | Nikkol MYS-1EX (Nikko), Coster K1 (Condea) | 2 |
| PEG-2 stearate | Nikkol MYS-2 (Nikko) | 4 |
| PEG-2 oleate | Nikkol MYO-2 (Nikko) | 4.5 |
| PEG-4 laurate | Mapeg ® 200 ML (PPG), Kessco ® PEG | 9.3 |
| | 200 ML (Stepan), LIPOPEG 2 L(LIPO Chem.) | |
| PEG-4 oleate | Mapeg ® 200 MO (PPG), Kessco ® PEG | 8.3 |
| | 200 MO (Stepan) | |
| PEG-4 stearate | Kessco ® PEG 200 MS (Stepan), Hodag | 6.5 |
| | 20 S (Calgene), Nikkol MYS-4 (Nikko) | |
| PEG-5 stearate | Nikkol TMGS-5 (Nikko) | 9.5 |
| PEG-5 oleate | Nikkol TMGO-5 (Nikko) | 9.5 |
| PEG-6 oleate | Algon OL 60 (Auschem SpA), Kessco ® | 8.5 |
| | PEG 300 MO (Stepan), Nikkol MYO-6 | |
| | (Nikko), Emulgante A6 (Condea) | |
| PEG-7 oleate | Algon OL 70 (Auschem SpA) | 10.4 |
| PEG-6 laurate | Kessco ® PEG300 ML (Stepan) | 11.4 |
| PEG-7 laurate | Lauridac 7 (Condea) | 13 |
| PEG-6 stearate | Kessco ® PEG300 MS (Stepan) | 9.7 |
| PEG-8 laurate | Mapeg ® 400 ML (PPG), | 13 |
| | LIPOPEG 4DL(Lipo Chem.) | |
| PEG-8 oleate | Mapeg ® 400 MO (PPG), Emulgante A8 (Condea) | 12 |
| PEG-8 stearate | Mapeg ® 400 MS (PPG), Myrj 45 | 12 |
| PEG-9 oleate | Emulgante A9 (Condea) | >10 |
| PEG-9 stearate | Cremophor S9 (BASF) | >10 |
| PEG-10 laurate | Nikkol MYL-10 (Nikko), Lauridac 10 (Croda) | 13 |
| PEG-10 oleate | Nikkol MYO-10 (Nikko) | 11 |
| PEG-12 stearate | Nikkol MYS-10 (Nikko), Coster K100 (Condea) | 11 |
| PEG-12 laurate | Kessco ® PEG 600 ML (Stepan) | 15 |
| PEG-12 oleate | Kessco ® PEG 600 MO (Stepan) | 14 |
| PEG-12 ricinoleate | (CAS #9004-97-1) | >10 |
| PEG-12 stearate | Mapeg ® 600 MS (PPG), Kessco ® PEG | 14 |
| | 600 MS (Stepan) | |
| PEG-15 stearate | Nikkol TMGS-15 (Nikko), Koster K15 (Condea) | 14 |
| PEG-15 oleate | Nikkol TMGO-15 (Nikko) | 15 |
| PEG-20 laurate | Kessco ® PEG 1000 ML (Stepan) | 17 |
| PEG-20 oleate | Kessco ® PEG 1000 MO (Stepan) | 15 |
| PEG-20 stearate | Mapeg ® 1000 MS (PPG), Kessco ® | 16 |
| | PEG 1000 MS (Stepan), Myrj 49 | |
| PEG-25 stearate | Nikkol MYS-25 (Nikko) | 15 |
| PEG-32 laurate | Kessco ® PEG 1540 ML (Stepan) | 16 |
| PEG-32 oleate | Kessco ® PEG 1540 MO (Stepan) | 17 |
| PEG-32 stearate | Kessco ® PEG 1540 MS (Stepan) | 17 |
| PEG-30 stearate | Myrj 51 | >10 |
| PEG-40 laurate | Crodet L40 (Croda) | 17.9 |
| PEG-40 oleate | Crodet O40 (Croda) | 17.4 |
| PEG-40 stearate | Myrj 52, Emerest ® 2715 (Henkel), | >10 |
| | Nikkol MYS-40 (Nikko) | |
| PEG-45 stearate | Nikkol MYS-45 (Nikko) | 18 |
| PEG-50 stearate | Myrj 53 | >10 |
| PEG-55 stearate | Nikkol MYS-55 (Nikko) | 18 |
| PEG-100 oleate | Crodet O-100 (Croda) | 18.8 |
| PEG-100 stearate | Myrj 59, Ariacel 165 (ICI) | 19 |
| PEG-200 oleate | Albunol 200 MO (Taiwan Surf.) | >10 |
| PEG-400 oleate | LACTOMUL (Henkel), Albunol 400 | >10 |
| | MO (Taiwan Surf.) | |
| PEG-600 oleate | Albunol 600 MO (Taiwan Surf.) | >10 |

2.2 PEG-Fatty Acid Diesters

Polyethylene glycol fatty acid diesters are also suitable for use as surfactants in the compositions of the present invention. Among the surfactants in Table 2, preferred hydrophilic surfactants include PEG-20 dilaurate, PEG-20 dioleate, PEG-20 distearate, PEG-32 dilaurate and PEG-32 dioleate. Representative PEG-fatty acid diesters are shown in Table 3.

TABLE 3
PEG-Fatty Acid Diester Surfactants

| COMPOUND | COMMERCIAL PRODUCT (Supplier) | HLB |
|---|---|---|
| PEG-4 dilaurate | Mapeg ® 200 DL (PPG), Kessco ® PEG | 7 |
| | 200 DL (Stepan), LIPOPEG 2-DL | |
| | (Lipo Chem.) | |
| PEG-4 dioleate | Mapeg ® 200 DO (PPG), | 6 |
| PEG-4 distearate | Kessco ® 200 DS (Stepan) | 5 |
| PEG-6 dilaurate | Kessco ® PEG 300 DL (Stepan) | 9.8 |
| PEG-6 dioleate | Kessco ® PEG 300 DO (Stepan) | 7.2 |
| PEG-6 distearate | Kessco ® PEG 300 DS (Stepan) | 6.5 |
| PEG-8 dilaurate | Mapeg ® 400 DL (PPG), Kessco ® PEG | 11 |
| | 400 DL (Stepan), LIPOPEG 4 DL | |
| | (Lipo Chem.) | |
| PEG-8 dioleate | Mapeg ® 400 DO (PPG), Kessco ® PEG | 8.8 |
| | 400 DO (Stepan), LIPOPEG 4 DO | |
| | (Lipo Chem.) | |
| PEG-8 distearate | Mapeg ® 400 DS (PPG), CDS 400 | 11 |
| | (Nikkol) | |
| PEG-10 dipalmitate | Polyaldo 2PKFG | >10 |
| PEG-12 dilaurate | Kessco ® PEG 600 DL (Stepan) | 11.7 |
| PEG-12 distearate | Kessco ® PEG 600 DS (Stepan) | 10.7 |
| PEG-12 dioleate | Mapeg ® 600 DO (PPG), Kessco ® 600 | 10 |
| | DO (Stepan) | |
| PEG-20 dilaurate | Kessco ® PEG 1000 DL (Stepan) | 15 |
| PEG-20 dioleate | Kessco ® PEG 1000 DO (Stepan) | 13 |
| PEG-20 distearate | Kessco ® PEG 1000 DS (Stepan) | 12 |
| PEG-32 dilaurate | Kessco ® PEG 1540 DL (Stepan) | 16 |
| PEG-32 dioleate | Kessco ® PEG 1540 DO (Stepan) | 15 |
| PEG-32 distearate | Kessco ® PEG 1540 DS (Stepan) | 15 |
| PEG-400 dioleate | Cithrol 4DO series (Croda) | >10 |
| PEG-400 distearate | Cithrol 4DS series (Croda) | >10 |

2.3 PEG-Fatty Acid Mono- and Di-ester Mixtures

In general, mixtures of surfactants are also useful in the present invention, including mixtures of two or more commercial surfactant products. Several PEG-fatty acid esters are marketed commercially as mixtures or mono- and diesters. Representative surfactant mixtures are shown in Table 4.

TABLE 4
PEG-Fatty Acid Mono- and Diester Mixtures

| | | |
|---|---|---|
| | COMMERCIAL | |
| COMPOUND | PRODUCT (Supplier) | HLB |

| | |
|---|---|
| PEG 4-150 mono, dilaurate | Kessco ® PEG 200-6000 mono, |
| | dilaurate (Stepan) |
| PEG 4-150 mono, dioleate | Kessco ® PEG 200-6000 mono, |
| | dioleate (Stepan) |
| PEG 4-150 mono, distearate | Kessco ® 200-6000 mono, |
| | distearate (Stepan) |

2.4 Polyethylene Glycol Glycerol Fatty Acid Esters

Suitable PEG glycerol fatty acid esters are shown in Table 5. Among the surfactants in the Table, preferred hydrophilic surfactants are PEG-20 glyceryl laurate, PEG-30 glyceryl laurate, PEG-40 glyceryl laurate, PEG-20 glyceryl oleate, and PEG-30 glyceryl oleate.

TABLE 5
PEG Glycerol Fatty Acid Esters

| | COMMERCIAL PRODUCT | |
|---|---|---|
| COMPOUND | (Supplier) | HLB |
| PEG-20 glyceryl laurate | Tagat ® L (Goldschmidt) | 16 |
| PEG-30 glyceryl laurate | Tagat ® L2 (Goldschmidt) | 16 |
| PEG-15 glyceryl laurate | Glycerox L series (Croda) | 15 |
| PEG-40 glyceryl laurate | Glycerox L series (Croda) | 15 |
| PEG-20 glyceryl stearate | Capmul ® EMG (ABITEC), | 13 |
| | Aldo ® MS-20 KFG (Lonza) | |
| PEG-20 glyceryl oleate | Tagat ® O (Goldschmidt) | >10 |
| PEG-30 glyceryl oleate | Tagat ® O2 (Goldschmidt) | >10 |

2.5. Alcohol-Oil Transesterification Products

A large number of surfactants of different degrees of lipophilicity or hydrophilicity can be prepared by reaction of alcohols or polyalcohols with a variety of natural and/or hydrogenated oils. Most commonly, the oils used are castor oil or hydrogenated castor oil, or an edible vegetable oil such as corn oil, olive oil, peanut oil, palm kernel oil, apricot kernel oil, or almond oil. Preferred alcohols include glycerol, propylene glycol, ethylene glycol, polyethylene glycol, sorbitol, and pentaerythritol. Among these alcohol-oil transesterified surfactants, preferred hydrophilic surfactants are PEG-35 castor oil (Incrocas-35), PEG-40 hydrogenated castor oil (Cremophor RH 40), PEG-25 trioleate (TAGAT® TO), PEG-60 corn glycerides (Crovol M70), PEG-60 almond oil (Crovol A70), PEG-40 palm kernel oil (Crovol PK70), PEG-50 castor oil (Emalex C-50), PEG-50 hydrogenated castor oil (Emalex HC-50), PEG-8 caprylickcapric glycerides (Labrasol), and PEG-6 caprylic/capric glycerides (Softigen 767). Preferred lipophilic surfactants in this class include PEG-5 hydrogenated castor oil, PEG-7 hydrogenated castor oil, PEG-9 hydrogenated castor oil, PEG-6 corn oil (Labrafil® M 2125 CS), PEG-6 almond oil (Labrafil® M 1966 CS), PEG-6 apricot kernel oil (Labrafil® M 1944 CS), PEG-6 olive oil (Labrafil® M 1980 CS), PEG-6 peanut oil (Labrafil® M 1969 CS), PEG-6 hydrogenated palm kernel oil (Labrafil® 2130 BS), PEG-6 palm kernel oil (Labrafil® M 2130 CS), PEG-6 triolein (Labrafil® M 2735 CS), PEG-8 corn oil (Labrafil® WL 2609 BS), PEG-20 corn glycerides (Crovol M40), and PEG-20 almond glycerides (Crovol A40). The latter two surfactants are reported to have HLB values of 10, which is generally considered to be the approximate border line between hydrophilic and lipophilic surfactants. For purposes of the present invention, these two surfactants are considered to be lipophilic.

Representative surfactants of this class suitable for use in the present invention are shown in Table 6.

TABLE 6
Transesterification Products of Oils and Alcohols

| COMPOUND | COMMERCIAL PRODUCT (Supplier) | HLB |
|---|---|---|
| PEG-3 castor oil | Nikkol CO-3 (Nikko) | 3 |
| PEG-5, 9, and 16 castor oil | ACCONON CA series (ABITEC) | 6-7 |
| PEG-20 castor oil | Emalex C-20 (Nihon Emulsion), Nikkol CO-20 TX | 11 |
| | (Nikko) | |
| PEG-23 castor oil | Emulgante EL23 | >10 |
| PEG-30 castor oil | Emalex C-30 (Nihon Emulsion), Alkamuls ® EL | 11 |
| | 620 (Rhone-Poulenc), Incrocas 30 (Croda) | |
| PEG-35 castor oil | Cremophor EL and EL-P (BASF), Emulphor EL, | |
| | Incrocas-35(Croda), Emulgin RO 35 (Henkel) | |
| PEG-38 castor oil | Emulgante EL 65 (Condea) | |
| PEG-40 castor oil | Emalex C-40 (Nihon Emulsion), Alkamuls ® EL | 13 |
| | 719 (Rhone-Poulenc) | |
| PEG-50 castor oil | Emalex C-50 (Nihon Emulsion) | 14 |
| PEG-56 castor oil | Eumulgin ® PRT 56 (Pulcra SA) | >10 |
| PEG-60 castor oil | Nikkol CO-60TX (Nikko) | 14 |
| PEG-100 castor oil | Thornley | >10 |
| PEG-200 castor oil | Eumulgin ® PRT 200 (Pulcra SA) | >10 |
| PEG-5 hydrogenated castor oil | Nikkol HCO-5 (Nikko) | 6 |
| PEG-7 hydrogenated castor oil | Simusol ® 989 (Seppic), Cremophor WO7 | 6 |
| | (BASF) | |
| PEG-10 hydrogenated castor oil | Nikkol HCO-10 (Nikko) | 6.5 |
| PEG-20 hydrogenated castor oil | Nikkol HCO-20 (Nikko) | 11 |
| PEG-25 hydrogenated castor oil | Simulsol ® 1292 (Seppic), Cerex ELS 250 | 11 |
| | (Auschem SpA) | |
| PEG-30 hydrogenated castor oil | Nikkol HCO-30 (Nikko) | 11 |
| PEG-40 hydrogenated castor oil | Cremophor RH 40 (BASF), Croduret (Croda), | 13 |
| | Emulgin HRE 40 | |
| | (Henkel) | |
| PEG-45 hydrogenated castor oil | Cerex ELS 450 (Auschem Spa) | 14 |
| PEG-50 hydrogenated castor oil | Emalex HC-50 (Nihon Emulsion) | 14 |
| PEG-60 hydrogenated castor oil | Nikkol HCO-60 (Nikko); Cremophor RH 60 | 15 |
| | (BASF) | |
| PEG-80 hydrogenated castor oil | Nikkol HCO-80 (Nikko) | 15 |
| PEG-100 hydrogenated castor oil | Nikkol HCO-100 (Nikko) | 17 |
| PEG-6 corn oil | Labrafil ® M 2125 CS (Gattefosse) | 4 |
| PEG-6 almond oil | Labrafil ® M 1966 CS (Gattefosse) | 4 |
| PEG-6 apricot kernel oil | Labrafil ® M 1944 CS (Gattefosse) | 4 |
| PEG-6 olive oil | Labrafil ® M 1980 CS (Gattefosse) | 4 |
| PEG-6 peanut oil | Labrafil ® M 1969 CS (Gattefosse) | 4 |
| PEG-6 hydrogenated palm kernel oil | Labrafil ® M 2130 BS (Gattefosse) | 4 |
| PEG-6 palm kernel oil | Labrafil ® M 2130 CS (Gattefosse) | 4 |
| PEG-6 triolein | Labrafil ® M 2735 CS (Gattefosse) | 4 |
| PEG-8 corn oil | Labrafil ® WL 2609 BS (Gattefosse) | 6-7 |
| PEG-20 corn glycerides | Crovol M40 (Croda) | 10 |
| PEG-20 almond glycerides | Crovol A40 (Croda) | 10 |
| PEG-25 trioleate | TAGAT ® TO (Goldschmidt) | 11 |
| PEG-40 palm kernel oil | Crovol PK-70 | >10 |
| PEG-60 corn glycerides | Crovol M70(Croda) | 15 |
| PEG-60 almond glycerides | Crovol A70 (Croda) | 15 |
| PEG-4 caprylic/capric triglyceride | Labrafac ® Hydro (Gattefosse), | 4-5 |
| PEG-8 caprylic/capric glycerides | Labrasol (Gattefosse), Labrafac ® CM 10 | >10 |
| | (Gattefosse) | |
| PEG-6 caprylic/capric glycerides | SOFTIGEN ® 767 (Huls), Glycerox 767 (Croda) | 19 |
| Lauroyl macrogol-32 glyceride | GELUCIRE 44/14 (Gattefosse) | 14 |
| Stearoyl macrogol glyceride | GELUCIRE 50/13 (Gattefosse) | 13 |
| Mono, di, tri, tetra esters of vegetable | SorbitoGlyceride (Gattefosse) | <10 |
| oils and sorbitol | | |
| Pentaerythrityl tetraisostearate | Crodamol PTIS (Croda) | <10 |
| Pentaerythrityl distearate | Albunol DS (Taiwan Surf.) | <10 |
| Pentaerythrityl tetraoleate | Liponate PO-4 (Lipo Chem.) | <10 |
| Pentaerythrityl tetrastearate | Liponate PS-4 (Lipo Chem.) | <10 |
| Pentaerythrityl tetracaprylate/ | Liponate PE-810 (Lipo Chem.), Crodamol PTC | <10 |
| tetracaprate | (Croda) | |
| Pentaerythrityl tetraoctanoate | Nikkol Pentarate 408 (Nikko) | |

Also included as oils in this category of surfactants are oil-soluble vitamins, such as vitamins A, D, E, K, etc. Thus, derivatives of these vitamins, such as tocopheryl PEG-1000 succinate (TPGS, available from Eastman), are also suitable surfactants.

2.6. Polyglycerized Fatty Acids

Polyglycerol esters of fatty acids are also suitable surfactants for the present invention. Among the polyglyceryl fatty acid esters, preferred lipophilic surfactants include polyglyceryl oleate (Plurol Oleique), polyglyceryl-2 dioleate (Nikkol DGDO), and polyglyceryl-10 trioleate. Preferred hydrophilic surfactants include polyglyceryl-110 laurate (Nikkol Decaglyn 1-L), polyglyceryl-10 oleate (Nikkol Decaglyn 1-O), and polyglyceryl-10 mono, dioleate (Caprol® PEG 860). Polyglyceryl polyricinoleates (Polymuls) are also preferred hydrophilic and lipophilic surfactants. Examples of suitable polyglyceryl esters are shown in Table 7.

TABLE 7
Polyglycerized Fatty Acids

| | COMMERCIAL | |
|---|---|---|
| COMPOUND | PRODUCT (Supplier) | HLB |
| Polyglyceryl-2 stearate | Nikkol DGMS (Nikko) | 5-7 |
| Polyglyceryl-2 oleate | Nikkol DGMO (Nikko) | 5-7 |
| Polyglyceryl-2 isostearate | Nikkol DGMIS (Nikko) | 5-7 |
| Polyglyceryl-3 oleate | Caprol ® 3GO (ABITEC), | 6.5 |
| | Drewpol 3-1-O (Stepan) | |
| Polyglyceryl-4 oleate | Nikkol Tetraglyn 1-O (Nikko) | 5-7 |
| Polyglyceryl-4 stearate | Nikkol Tetraglyn 1-S (Nikko) | 5-6 |
| Polyglyceryl-6 oleate | Drewpol 6-1-O (Stepan), Nikkol | 9 |
| | Hexaglyn 1-O (Nikko) | |
| Polyglyceryl-10 laurate | Nikkol Decaglyn 1-L (Nikko) | 15 |
| Polyglyceryl-10 oleate | Nikkol Decaglyn 1-O (Nikko) | 14 |
| Polyglyceryl-10 stearate | Nikkol Decaglyn 1-S (Nikko) | 12 |
| Polyglyceryl-6 ricinoleate | Nikkol Hexaglyn PR-15 (Nikko) | >8 |
| Polyglyceryl-10 linoleate | Nikkol Decaglyn 1-LN (Nikko) | 12 |
| Polyglyceryl-6 pentaoleate | Nikkol Hexaglyn 5-O (Nikko) | <10 |
| Polyglyceryl-3 dioleate | Cremophor GO32 (BASF) | <10 |
| Polyglyceryl-3 distearate | Cremophor GS32 (BASF) | <10 |
| Polyglyceryl-4 pentaoleate | Nikkol Tetraglyn 5-O (Nikko) | <10 |
| Polyglyceryl-6 dioleate | Caprol ® 6G20 (ABITEC); | 8.5 |
| | Hodag PGO-62 (Calgene), | |
| | PLUROL OLEIQUE CC 497 | |
| | (Gattefosse) | |
| Polyglyceryl-2 dioleate | Nikkol DGDO (Nikko) | 7 |
| Polyglyceryl-10 trioleate | Nikkol Decaglyn 3-O (Nikko) | 7 |
| Polyglyceryl-10 pentaoleate | Nikkol Decaglyn 5-O (Nikko) | 3.5 |
| Polyglyceryl-10 septaoleate | Nikkol Decaglyn 7-O (Nikko) | 3 |
| Polyglyceryl-10 tetraoleate | Caprol ® 10G4O (ABITEC); | 6.2 |
| | Hodag PGO-62 (CALGENE), | |
| | Drewpol 10-4-O (Stepan) | |
| Polyglyceryl-10 decaisostearate | Nikkol Decaglyn 10-IS (Nikko) | <10 |
| Polyglyceryl-101 decaoleate | Drewpol 10-10-O (Stepan), | 3.5 |
| | Caprol 10G10O (ABITEC), | |
| | Nikkol Decaglyn 10-O | |
| Polyglyceryl-10 mono, dioleate | Caprol ® PGE 860 (ABITEC) | 11 |
| Polyglyceryl polyricinoleate | Polymuls (Henkel) | 3-20 |

2.7. Propylene Glycol Fatty Acid Esters

Esters of propylene glycol and fatty acids are suitable surfactants for use in the present invention. In this surfactant class, preferred lipophilic surfactants include propylene glycol monolaurate (Lauroglycol FCC), propylene glycol ricinoleate (Propymuls), propylene glycol monooleate (Myverol P-O6), propylene glycol dicaprylate/dicaprate (Captex ® 200), and propylene glycol dioctanoate (Captex® 800). Examples of surfactants of this class are given in Table 8.

TABLE 8
Propylene Glycol Fatty Acid Esters

| | COMMERCIAL | |
|---|---|---|
| COMPOUND | PRODUCT (Supplier) | HLB |
| Propylene glycol monocaprylate | Capryol 90 (Gattefosse), | <10 |
| | Nikkol Sefsol 218 (Nikko) | |
| Propylene glycol monolaurate | Lauroglycol 90 (Gattefosse), | <10 |
| | Lauroglycol FCC (Gattefosse) | |
| Propylene glycol oleate | Lutrol OP2000 (BASF) | <10 |
| Propylene glycol myristate | Mirpyl | <10 |
| Propylene glycol monostearate | ADM PGME-03 (ADM), LIPO | 3-4 |
| | PGMS (Lipo Chem.), | |
| | Aldo ® PGHMS (Lonza) | |
| Propylene glycol hydroxy stearate | | <10 |
| Propylene glycol ricinoleate | PROPYMULS (Henkel) | <10 |
| Propylene glycol isostearate | | <10 |
| Propylene glycol monooleate | Myverol P-O6 (Eastman) | <10 |
| Propylene glycol dicaprylate/dicaprate | Captex ® 200 (ABITEC), Miglyol ® | >6 |
| | 840 (Huls), Neobee ® M-20 (Stepan) | |
| Propylene glycol dioctanoate | Captex ® 800 (ABITEC) | >6 |
| Propylene glycol caprylate/caprate | LABRAFAC PG (Gattefosse) | >6 |
| Propylene glycol dilaurate | | >6 |
| Propylene glycol distearate | Kessco ® PGDS (Stepan) | >6 |
| Propylene glycol dicaprylate | Nikkol Sefsol 228 (Nikko) | >6 |
| Propylene glycol dicaprate | Nikkol PDD (Nikko) | >6 |

2.8. Mixtures of Propylene Glycol Esters-Glycerol Esters

In general, mixtures of surfactants are also suitable for use in the present invention. In particular, mixtures of propylene glycol fatty acid esters and glycerol fatty acid esters are suitable and are commercially available. One preferred mixture is composed of the oleic acid esters of propylene glycol and glycerol (Arlacel 186). Examples of these surfactants are shown in Table 9.

TABLE 9
Glycerol/Propylene Glycol Fatty Acid Esters

| COMPOUND | COMMERCIAL PRODUCT (Supplier) | HLB |
|---|---|---|
| Oleic | ATMOS 300, ARLACEL 186 (ICI) | 3-4 |
| Stearic | ATMOS 150 | 3-4 |

2.9. Mono- and Diglycerides

A particularly important class of surfactants is the class of mono- and diglycerides. These surfactants are generally lipophilic. Preferred lipophilic surfactants in this class of compounds include glyceryl monooleate (Peceol), glyceryl ricinoleate, glyceryl laurate, glyceryl dilaurate (Capmul® GDL), glyceryl dioleate (Capmul® GDO), glyceryl mono/dioleate (Capmul® GMO-K), glyceryl caprylatelcaprate (Capmul® MCM), caprylic acid mono/diglycerides (Imwitor® 988), and mono- and diacetylated monoglycerides (Myvacet® 9-45). Examples of these surfactants are given in Table 10.

TABLE 10
Mono- and Diglyceride Surfactants

| | COMMERCIAL | |
|---|---|---|
| COMPOUND | PRODUCT (Supplier) | HLB |
| Monopalmitolein (C16:1) | (Larodan) | <10 |
| Monoelaidin (C18:1) | (Larodan) | <10 |
| Monocaproin (C6) | (Larodan) | <10 |
| Monocaprylin | (Larodan) | <10 |
| Monocaprin | (Larodan) | <10 |
| Monolaurin | (Larodan) | <10 |
| Glyceryl monomyristate (C14) | Nikkol MGM (Nikko) | 3-4 |
| Glyceryl monooleate (C18:1) | PECEOL (Gattefosse), Hodag | 3-4 |
| | GMO-D, Nikkol MGO (Nikko) | |
| Glyceryl monooleate | RYLO series (Danisco), | 3-4 |
| | DIMODAN series (Danisco), | |
| | EMULDAN (Danisco), ALDO ® | |
| | MO FG (Lonza), Kessco ® GMO | |
| | (Stepan), MONOMULS ® series | |
| | (Henkel), TEGIN O, | |
| | DREWMULSE GMO (Stepan), | |
| | Atlas G-695 (ICI), GMOrphic 80 | |
| | (Eastman), ADM DMG-40, 70, | |
| | and 100 (ADM), Myverol(Eastman) | |
| Glycerol monooleate/linoleate | OLICINE (Gattefosse) | 3-4 |
| Glycerol monolinoleate | Maisine (Gattefosse), MYVEROL | 3-4 |
| | 18-92, Myverol 18-06 (Eastman) | |
| Glyceryl ricinoleate | Softigen ® 701 (Huls), | 6 |
| | HODAG GMR-D (Calgene), | |
| | ALDO ® MR (Lonza) | |
| Glyceryl monolaurate | ALDO ® MLD (Lonza), | 6.8 |
| | Hodag GML (Calgene) | |
| Glycerol monopalmitate | Emalex GMS-P (Nihon) | 4 |
| Glycerol monostearate | Capmul ® GMS (ABITEC), | 5-9 |
| | Myvaplex (Eastman), | |
| | IMWITOR ® 191 (Huls), | |
| | CUTINA GMS, Aldo ® MS | |
| | (Lonza), Nikkol MGS series(Nikko) | |
| Glyceryl mono-, dioleate | Capmul ® GMO-K (ABITEC) | <10 |
| Glyceryl palmitic/stearic | CUTINA MD-A, ESTAGEL-G18 | <10 |
| Glyceryl acetate | Lamegin ® EE (Grunau GmbH) | <10 |
| Glyceryl laurate | Imwitor ® 312 (Huls), | 4 |
| | Monomuls ® 90-45 (Grunau | |
| | GmbH), Aldo ® MLD (Lonza) | |
| Glyceryl citrate/lactate/oleate/linoleate | Imwitor ® 375 (Huls) | <10 |
| Glyceryl caprylate | Imwitor ® 308 (Huls), | 5-6 |
| | Capmul ® MCMC8 (ABITEC) | |
| Glyceryl caprylate/caprate | Capmul ® MCM (ABITEC) | 5-6 |
| Caprylic acid mono, diglycerides | Imwitor ® 988 (Huls) | 5-6 |
| Caprylic/capric glycerides | Imwitor ® 742 (Huls) | <10 |
| Mono- and diacetylated | Myvacet ® 9-45, Myvacet ® | 3.8-4 |
| monoglycerides | 9-40, Myvacet ® 9-08 (Eastman), | |
| | Lamegin ® (Grunau) | |
| Glyceryl monostearate | Aldo ® MS, Arlacel 129 (ICI), | 4.4 |
| | LIPO GMS (Lipo Chem.), | |
| | Imwitor ® 191 (Huls), | |
| | Myvaplex (Eastman) | |
| Lactic acid esters of | LAMEGIN GLP (Henkel) | <10 |
| mono, diglycerides | | |
| Dicaproin (C6) | (Larodan) | <10 |
| Dicaprin (C10) | (Larodan) | <10 |
| Dioctanoin (C8) | (Larodan) | <10 |
| Dimyristin (C14) | (Larodan) | <10 |
| Dipalmitin (C16) | (Larodan) | <10 |
| Distearin | (Larodan) | <10 |
| Glyceryl dilaurate (C12) | Capmul ® GDL (ABITEC) | 3-4 |
| Glyceryl dioleate | Capmul ® GDO (ABITEC) | 3-4 |
| Glycerol esters of fatty acids | GELUCIRE 39/01 (Gattefosse), | 1 |
| | GELUCIRE 43/01 (Gattefosse) | 6 |
| | GELUCIRE 37/06 (Gattefosse) | |
| Dipalmitolein (C16:1) | (Larodan) | <10 |
| 1,2 and 1,3-diolein (C18:1) | (Larodan) | <10 |
| Dielaidin (C18:1) | (Larodan) | <10 |
| Dilinolein (C18:2) | (Larodan) | <10 |

2.10. Sterol and Sterol Derivatives

Sterols and derivatives of sterols are suitable surfactants for use in the present invention. These surfactants can be hydrophilic or lipophilic. Preferred derivatives include the polyethylene glycol derivatives. A preferred lipophilic surfactant in this class is cholesterol. A preferred hydrophilic surfactant in this class is PEG-24 cholesterol ether Solulan C-24). Examples of surfactants of this class are shown in Table 11.

TABLE 11
Sterol and Sterol Derivative Surfactants

| | COMMERCIAL PRODUCT | |
|---|---|---|
| COMPOUND | (Supplier) | HLB |
| Cholesterol, sitosterol, lanosterol | | <10 |
| PEG-24 cholesterol ether | Solulan C-24 (Amerchol) | >10 |
| PEG-30 cholestanol | Nikkol DHC (Nikko) | >10 |
| Phytosterol | GENEROL series (Henkel) | <10 |
| PEG-25 phyto sterol | Nikkol BPSH-25 (Nikko) | >10 |
| PEG-5 soya sterol | Nikkol BPS-5 (Nikko) | <10 |
| PEG-10 soya sterol | Nikkol BPS-10 (Nikko) | <10 |
| PEG-20 soya sterol | Nikkol BPS-20 (Nikko) | <10 |
| PEG-30 soya sterol | Nikkol BPS-30 (Nikko) | >10 |

2.11. Polyethylene Glycol Sorbitan Fatty Acid Esters

A variety of PEG-sorbitan fatty acid esters are available and are suitable for use as surfactants in the present invention. In general, these surfactants are hydrophilic, although several lipophilic surfactants of this class can be used. Among the PEG-sorbitan fatty acid esters, preferred hydrophilic surfactants include PEG-20 sorbitan monolaurate (Tween-20), PEG-20 sorbitan monopalmitate (Tween-40), PEG-20 sorbitan monostearate (Tween-60), and PEG-20 sorbitan monooleate (Tween-80). Examples of these surfactants are shown in Table 12.

TABLE 12
PEG-Sorbitan Fatty Acid Esters

| | COMMERCIAL | |
|---|---|---|
| COMPOUND | PRODUCT (Supplier) | HLB |
| PEG-10 sorbitan laurate | Liposorb L-10 (Lipo Chem.) | >10 |
| PEG-20 sorbitan monolaurate | Tween-20 (Atlas/ICI), Crillet 1 | 17 |
| | (Croda), DACOL MLS 20 | |
| | (Condea) | |
| PEG-4 sorbitan monolaurate | Tween-21 (Atlas/ICI), Crillet | 13 |
| | 11 (Croda) | |
| PEG-80 sorbitan monolaurate | Hodag PSML-80 (Calgene); | >10 |
| | T-Maz 28 | |
| PEG-6 sorbitan monolaurate | Nikkol GL-1 (Nikko) | 16 |
| PEG-20 sorbitan | Tween-40 (Atlas/ICI), Crillet 2 | 16 |
| monopalmitate | (Croda) | |
| PEG-20 sorbitan monostearate | Tween-60 (Atlas/ICI), Crillet 3 | 15 |
| | (Croda) | |
| PEG-4 sorbitan monostearate | Tween-61 (Atlas/ICI), Crillet 31 | 9.6 |
| | (Croda) | |
| PEG-8 sorbitan monostearate | DACOL MSS (Condea) | >10 |
| PEG-6 sorbitan monostearate | Nikkol TS106 (Nikko) | 11 |
| PEG-20 sorbitan tristearate | Tween-65 (Atlas/ICI), Crillet 35 | 11 |
| | (Croda) | |
| PEG-6 sorbitan tetrastearate | Nikkol GS-6 (Nikko) | 3 |
| PEG-60 sorbitan tetrastearate | Nikkol GS-460 (Nikko) | 13 |
| PEG-5 sorbitan monooleate | Tween-81 (Atlas/ICI), Crillet 41 | 10 |
| | (Croda) | |
| PEG-6 sorbitan monooleate | Nikkol TO-106 (Nikko) | 10 |
| PEG-20 sorbitan monooleate | Tween-80 (Atlas/ICI), Crillet 4 | 15 |
| | (Croda) | |
| PEG-40 sorbitan oleate | Emalex ET 8040 (Nihon | 18 |
| | Emulsion) | |
| PBG-20 sorbitan trioleate | Tween-85 (Atlas/ICI), Crillet 45 | 11 |
| | (Croda) | |
| PEG-6 sorbitan tetraoleate | Nikkol GO-4 (Nikko) | 8.5 |
| PEG-30 sorbitan tetraoleate | Nikkol GO-430 (Nikko) | 12 |
| PEG-40 sorbitan tetraoleate | Nikkol GO-440 (Nikko) | 13 |
| PEG-20 sorbitan | Tween-120 (Atlas/ICI), Crillet 6 | >10 |
| monoisostearate | (Croda) | |
| PEG sorbitol hexaoleate | Atlas G-1086 (ICI) | 10 |
| PEG-6 sorbitol hexastearate | Nikkol GS-6 (Nikko) | 3 |

2.12. Polyethylene Glycol Alkyl Ethers

Ethers of polyethylene glycol and alkyl alcohols are suitable surfactants for use in the present invention. Preferred lipophilic ethers include PEG-3 oleyl ether (Volpo 3) and PEG-4 lauryl ether (Brij 30). Examples of these surfactants are shown in Table 13.

TABLE 13
Polyethylene Glycol Alkyl Ethers

| | COMMERCIAL | |
|---|---|---|
| COMPOUND | PRODUCT (Supplier) | HLB |
| PEG-2 oleyl ether,oleth-2 | Brij 92/93 (Atlas/ICI) | 4.9 |
| PEG-3 oleyl ether,oleth-3 | Volpo 3 (Croda) | <10 |
| PEG-5 oleyl ether,oleth-5 | Volpo 5 (Croda) | <10 |
| PEG-10 oleyl ether,oleth-10 | Volpo 10 (Croda), Brij | 12 |
| | 96/97 (Atlas/ICI) | |
| PEG-20 oleyl ether,oleth-20 | Volpo 20 (Croda), Brij 98/99 | 15 |
| | (Atlas/ICI) | |
| PEG-4 lauryl ether,laureth-4 | Brij 30 (Atlas/ICI) | 9.7 |
| PEG-9 lauryl ether | | >10 |
| PEG-23 lauryl ether,laureth-23 | Brij 35 (Atlas/ICI) | 17 |
| PEG-2 cetyl ether | Brij 52 (ICI) | 5.3 |
| PEG-10 cetyl ether | Brij 56 (ICI) | 13 |
| PEG-20 cetyl ether | Brij 58 (ICI) | 16 |
| PEG-2 stearyl ether | Brij 72 (ICI) | 4.9 |
| PEG-10 stearyl ether | Brij 76 (ICI) | 12 |
| PEG-20 stearyl ether | Brij 78 (ICI) | 15 |
| PEG-100 stearyl ether | Brij 700 (ICI) | >10 |

2.13. Sugar Esters

Esters of sugars are suitable surfactants for use in the present invention. Preferred hydrophilic surfactants in this class include sucrose monopalmitate and sucrose monolaurate. Examples of such surfactants are shown in Table 14.

TABLE 14
Sugar Ester Surfactants

| | COMMERCIAL PRODUCT | |
|---|---|---|
| COMPOUND | (Supplier) | HLB |
| Sucrose distearate | SUCRO ESTER 7 (Gattefosse), | 3 |
| | Crodesta F-10 (Croda) | |
| Sucrose distearate/monostearate | SUCRO ESTER 11 (Gattefosse), | 12 |
| | Crodesta F-110 (Croda) | |
| Sucrose dipalmitate | | 7.4 |
| Sucrose monostearate | Crodesta F-160 (Croda) | 15 |
| Sucrose monopalmitate | SUCRO ESTER 15 (Gattefosse) | >10 |
| Sucrose monolaurate | Saccharose monolaurate 1695 | 15 |
| | (Mitsubisbi-Kasei) | |

2.14. Polyethylene Glycol Alkyl Phenols

Several hydrophilic PEG-alkyl phenol surfactants are available, and are suitable for use in the present invention. Examples of these surfactants are shown in Table 15.

TABLE 15
Polyethylene Glycol Alkyl Phenol Surfactants

| | COMMERCIAL PRODUCT | |
|---|---|---|
| COMPOUND | (Supplier) | HLB |
| PEG-10-100 nonyl | Triton X series (Rohm & Haas), Igepal CA | >10 |
| phenol | series (GAF, USA), Antarox CA series | |
| | (GAF, UK) | |
| PEG-15-100 octyl | Triton N-series (Rohm & Haas), Igepal CO | >10 |
| phenol ether | series (GAF, USA), Antarox CO series | |
| | (GAF, UK) | |

2.15. Polyoxyethylene-Polyoxypropylene Block Copolymers

The POE-POP block copolymers are a unique class of polymeric surfactants.

The unique structure of the surfactants, with hydrophilic POE and lipophilic POP moieties in well-defined ratios and positions, provides a wide variety of surfactants suitable for use in the present invention. These surfactants are available under various trade names, including Synperonic PE series (ICI); Pluronic ® series (BASF), Emkalyx, Lutrol (BASF), Supronic, Monolan, Pluracare, and Plurodac. The generic term for these polymers is “poloxamer” (CAS 9003-11-6). These polymers have the formula:

HO(C<2>H<4>O)<a>(C<3>H<6>O)<b>(C<2>H<4>O)<a>H

where “a” and “b” denote the number of polyoxyethylene and polyoxypropylene units, respectively.

Preferred hydrophilic surfactants of this class include Poloxamers 108, 188, 217, 238, 288, 338, and 407. Preferred lipophilic surfactants in this class include Poloxamers 124, 182, 183, 212, 331, and 335.

Examples of suitable surfactants of this class are shown in Table 15. Since the compounds are widely available, commercial sources are not listed in the Table. The compounds are listed by generic name, with the corresponding “a” and “b” values.

TABLE 16
POE-POP Block Copolymers

| | a, b values in | | |
|---|---|---|---|
| | HO(C<2>H<4>O)<a> | | |
| | (C<3>H<6>O)<b>(C<2>H< | | |
| COMPOUND | 4>O)<a>H | | HLB |
| Poloxamer 105 | a 11 | b 16 | 8 |
| Poloxamer 108 | a 46 | b 16 | >10 |
| Poloxamer 122 | a 5 | b 21 | 3 |
| Poloxamer 123 | a 7 | b 21 | 7 |
| Poloxamer 124 | a 11 | b 21 | >7 |
| Poloxamer 181 | a 3 | b 30 | |
| Poloxamer 182 | a 8 | b 30 | 2 |
| Poloxamer 183 | a 10 | b 30 | |
| Poloxamer 184 | a 13 | b 30 | |
| Poloxamer 185 | a 19 | b 30 | |
| Poloxamer 188 | a 75 | b 30 | 29 |
| Poloxamer 212 | a 8 | b 35 | |
| Poloxamer 215 | a 24 | b 35 | |
| Poloxamer 217 | a 52 | b 35 | |
| Poloxamer 231 | a 16 | b 39 | |
| Poloxamer 234 | a 22 | b 39 | |
| Poloxamer 235 | a 27 | b 39 | |
| Poloxamer 237 | a 62 | b 39 | 24 |
| Poloxamer 238 | a 97 | b 39 | |
| Poloxamer 282 | a 10 | b 47 | |
| Poloxamer 284 | a 21 | b 47 | |
| Poloxamer 288 | a 122 | b 47 | >10 |
| Poloxamer 331 | a 7 | b 54 | 0.5 |
| Poloxamer 333 | a 20 | b 54 | |
| Poloxamer 334 | a 31 | b 54 | |
| Poloxamer 335 | a 38 | b 54 | |
| Poloxamer 338 | a 128 | b 54 | |
| Poloxamer 401 | a 6 | b 67 | |
| Poloxamer 402 | a 13 | b 67 | |
| Poloxamer 403 | a 21 | b 67 | |
| Poloxamer 407 | a 98 | b 67 | |

2.16. Sorbitan Fatty Acid Esters

Sorbitan esters of fatty acids are suitable surfactants for use in the present invention. Among these esters, preferred lipophilic surfactants include sorbitan monolaurate (Arlacel 20), sorbitan monopalmitate (Span-40), sorbitan monooleate (Span-80), sorbitan monostearate, and sorbitan tristearate. Examples of these surfactants are shown in Table 17.

TABLE 17
Sorbitan Fatty Acid Ester Surfactants

| | COMMERCIAL PRODUCT | |
|---|---|---|
| COMPOUND | (Supplier) | HLB |
| Sorbitan monolaurate | Span-20 (Atlas/ICI), Crill 1 (Croda), | 8.6 |
| | Arlacel 20 (ICI) | |
| Sorbitan monopalmitate | Span-40 (Atlas/ICI), Crill 2 (Croda), | 6.7 |
| | Nikkol SP-10 (Nikko) | |
| Sorbitan monooleate | Span-80 (Atlas/ICI), Crill 4 (Croda), | 4.3 |
| | Crill 50 (Croda) | |
| Sorbitan monostearate | Span-60 (Atlas/ICI), Crill 3 (Croda), | 4.7 |
| | Nikkol SS-10 (Nikko) | |
| Sorbitan trioleate | Span-85 (Atlas/ICI), Crill 45 (Croda), | 4.3 |
| | Nikkol SO-30 (Nikko) | |
| Sorbitan sesquioleate | Arlacel-C (ICI), Crill 43 (Croda), | 3.7 |
| | Nikkol SO-15 (Nikko) | |
| Sorbitan tristearate | Span-65 (Atlas/ICI) Crill 35 (Croda), | 2.1 |
| | Nikkol SS-30 (Nikko) | |
| Sorbitan monoisostearate | Crill 6 (Croda), Nikkol SI-10 (Nikko) | 4.7 |
| Sorbitan sesquistearate | Nikkol SS-15 (Nikko) | 4.2 |

2.17. Lower Alcohol Fatty Acid Esters

Esters of lower alcohols (C<2> to C<4>) and fatty acids (C<8> to C<18>) are suitable surfactants for use in the present invention. Among these esters, preferred lipophilic surfactants include ethyl oleate (Crodamol EO), isopropyl myristate (Crodamol IPM), and isopropyl palmitate (Crodamol IPP). Examples of these surfactants are shown in Table 18.

TABLE 18
Lower Alcohol Fatty Acid Ester Surfactants

| | COMMERCIAL PRODUCT | |
|---|---|---|
| COMPOUND | (Supplier) | HLB |
| Ethyl oleate | Crodamol EO (Croda), Nikkol EOO (Nikko) | <10 |
| Isopropyl myristate | Crodamol IPM (Croda) | <10 |
| Isopropyl palmitate | Crodamol IPP (Croda) | <10 |
| Ethyl linoleate | Nikkol VF-E (Nikko) | <10 |
| Isopropyl linoleate | Nikkol VF-IP (Nikko) | <10 |

2.18. Ionic Surfactants

Ionic surfactants, including cationic, anionic and zwitterionic surfactants, are suitable hydrophilic surfactants for use in the present invention. Preferred anionic surfactants include fatty acid salts and bile salts. Specifically, preferred ionic surfactants include sodium oleate, sodium lauryl sulfate, sodium lauryl sarcosinate, sodium dioctyl sulfosuccinate, sodium cholate, and sodium taurocholate. Examples of such surfactants are shown in Table 18 below. For simplicity, typical counterions are shown in the entries in the Table. It will be appreciated by one skilled in the art, however, that any bioacceptable counterion may be used. For example, although the fatty acids are shown as sodium salts, other cation counterions can also be used, such as alkali metal cations or ammonium. Unlike typical non-ionic surfactants, these ionic surfactants are generally available as pure compounds, rather than commercial (proprietary) mixtures. Because these compounds are readily available from a variety of commercial suppliers, such as Aldrich, Sigma, and the like, commercial sources are not generally listed in Table 19.

TABLE 19
Ionic Surfactants

| COMPOUND | HLB |
|---|---|
| FATTY ACID SALTS | >10 |
| Sodium caproate | |
| Sodium caprylate | |
| Sodium caprate | |
| Sodium laurate | |
| Sodium myristate | |
| Sodium myristolate | |
| Sodium palmitate | |
| Sodium palmitoleate | |
| Sodium oleate | 18 |
| Sodium ricinoleate | |
| Sodium linoleate | |
| Sodium linolenate | |
| Sodium stearate | |
| Sodium lauryl sulfate (dodecyl) | 40 |
| Sodium tetradecyl sulfate | |
| Sodium lauryl sarcosinate | |
| Sodium dioctyl sulfosuccinate [sodium docusate (Cytec)] | |
| BILE SALTS | >10 |
| Sodium cholate | |
| Sodium taurocholate | |
| Sodium glycocholate | |
| Sodium deoxycholate | |
| Sodium taurodeoxycholate | |
| Sodium glycodeoxycholate | |
| Sodium ursodeoxycholate | |
| Sodium chenodeoxycholate | |
| Sodium taurochenodeoxycholate | |
| Sodium glyco cheno deoxycholate | |
| Sodium cholylsarcosinate | |
| Sodium N-methyl taurocholate | |
| PHOSPHOLIPIDS | |
| Egg/Soy lecithin [EpikuronTM (Lucas Meyer), | |
| OvothinTM] (Lucas Meyer)] | |
| Lyso egg/soy lecithin | |
| Hydroxylated lecithin | |
| Lysophosphatidylcholine | |
| Cardiolipin | |
| Sphingomyelin | |
| Phosphatidylcholine | |
| Phosphatidyl ethanolamine | |
| Phosphatidic acid | |
| Phosphatidyl glycerol | |
| Phosphatidyl serine | |
| PHOSPHORIC ACID ESTERS | |
| Diethanolammonium polyoxyethylene-10 oleyl ether phosphate | |
| Esterification products of fatty alcohols or fatty alcohol ethoxylates | |
| with phosphoric acid or anhydride | |
| CARBOXYLATES | |
| Ether carboxylates (by oxidation of terminal OH group of | |
| fatty alcohol ethoxylates) | |
| Succinylated monoglycerides [LAMEGIN ZB (Henkel)] | |
| Sodium stearyl fumarate | |
| Stearoyl propylene glycol hydrogen succinate | |
| Mono/diacetylated tartaric acid esters of mono- and diglycerides | |
| Citric acid esters of mono-, diglycerides | |
| Glyceryl-lacto esters of fatty acids (CFR ref. 172.852) | |
| Acyl lactylates: | |
| lactylic esters of fatty acids | |
| calcium/sodium stearoyl-2-lactylate | |
| calcium/sodium stearoyl lactylate | |
| Alginate salts | |
| Propylene glycol alginate | |
| SULFATES AND SULFONATES | |
| Ethoxylated alkyl sulfates | |
| Alkyl benzene sulfones | |
| α-olefin sulfonates | |
| Acyl isethionates | |
| Acyl taurates | |
| Alkyl glyceryl ether sulfonates | |
| Octyl sulfosuccinate disodium | |
| Disodium undecylenamideo-MEA-sulfosuccinate | |
| CATIONIC Surfactants | >10 |
| Hexadecyl triammonium bromide | |
| Decyl trimethyl ammonium bromide | |
| Cetyl trimethyl ammonium bromide | |
| Dodecyl ammonium chloride | |
| Alkyl benzyldimethylammonium salts | |
| Diisobutyl phenoxyethoxydimethyl benzylammonium salts | |
| Alkylpyridinium salts | |
| Betaines (trialkylglycine) | |
| Lauryl betaine (N-lauryl,N,N-dimethylglycine) | |
| Ethoxylated amines: | |
| Polyoxyethylene-15 coconut amine | |

2.20 Preferred Surfactants and Surfactant Combinations

Among the above-listed surfactants, several combinations are preferred. Preferred non-ionic hydrophilic surfactants include alkylglucosides; alkylmaltosides; alkylthioglucosides; lauryl macrogolglycerides; polyoxyethylene alkyl ethers; polyoxyethylene alkylphenols; polyethylene glycol fatty acids esters; polyethylene glycol glycerol fatty acid esters; polyoxyethylene sorbitan fatty acid esters; polyoxyethylene-polyoxypropylene block copolymers; polyglycerol fatty acid esters; polyoxyethylene glycerides; polyoxyethylene sterols, derivatives, and analogues thereof; polyoxyethylene vegetable oils; polyoxyethylene hydrogenated vegetable oils; reaction mixtures of polyols with fatty acids, glycerides, vegetable oils, hydrogenated vegetable oils, and sterols; sugar esters, sugar ethers; sucroglycerides; and mixtures thereof.

More preferably, the non-ionic hydrophilic surfactant is selected from the group consisting of polvoxyethylene alkylethers; polyethylene glycol fatty acids esters; polyethylene glycol glycerol fatty acid esters; polyoxyethylene sorbitan fatty acid esters; polyoxyethylene-polyoxypropylene block copolymers; polyglyceryl fatty acid esters; polyoxyethylene glycerides; polyoxyethylene vegetable oils; and polyoxyethylene hydrogenated vegetable oils. The glyceride can be a monoglyceride, diglyceride, triglyceride, or a mixture.

Also preferred are non-ionic hydrophilic surfactants that are reaction mixtures of polyols and fatty acids, glycerides, vegetable oils, hydrogenated vegetable oils or sterols. These reaction mixtures are largely composed of the transesterification products of the reaction, along with often complex mixtures of other reaction products. The polyol is preferably glycerol, ethylene glycol, polyethylene glycol, sorbitol, propylene glycol, pentaerythritol, or a saccharide.

Several particularly preferred carrier compositions are those which include as a non-ionic hydrophilic surfactant PEG-10 laurate, PEG-12 laurate, IIEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 triolcate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10 oleate, Tween 40, Tween 60, sucrose monostearate, sucrose monolaurate, sucrose monopalmitate, PEG 10-100 nonyl phenol series, PEG 15-100 octyl phenol series, or a poloxamer.

Among these preferred surfactants, more preferred are PEG-20 laurate, PEG-20 oleate, PEG-35 castor oil, PEG-40 palm kernel oil, PEG-40 hydrogenated castor oil, PEG-60 corn oil, PEG-25 glyceryl trioleate, polyglyceryl-10 laurate, PEG-6 caprate/caprylate glycerides, PEG-8 caprate/caprylate glycerides, PEG-30 cholesterol, polysorbate 20 polysorbate 80, POE-9 lauryl ether, POE-23 lauryl ether, POE-10 oleyl ether, PEG-24 cholesterol, sucrose monostearate, sucrose monolaurate and poloxamers. Most preferred are PEG-35 castor oil, PEG-40 hydrogenated castor oil, PEG-60 corn oil, PEG-25 glyceryl trioleate, PEG-6 caprate/caprylate glycerides, PEG-8 caprate/caprylate glycerides, polysorbate 20, polysorbate 80, tocopheryl PEG-1000 succinate, PEG-24 cholesterol, and hydrophilic poloxamers.

The hydrophilic surfactant can also be, or include as a component, an ionic surfactant. Preferred ionic surfactants include alkyl ammonium salts; bile acids and salts, analogues, and derivatives thereof; fusidic acid and derivatives thereof; fatty acid derivatives of amino acids, oligopeptides, and polypeptides; glyceride derivatives of amino acids oligopeptides, and polypeptides; acyl lactylates; mono-diacetylated tartaric acid esters of mono-diglycerides; succinylated monoglycerides; citric acid esters of mono-diglycerides; alginate salts; propylene glycol alginate; lecithins and hydrogenated lecithins; lysolecithin and hydrogenated lysolecithins; lysophospholipids and derivatives thereof; phospholipids and derivatives thereof; salts of alkylsulfates; salts of fatty acids; sodium docusate; carnitines; and mixtures thereof. More preferable ionic surfactants include bile acids and salts, analogues, and derivatives thereof; lecithins, lysolecithin, phospholipids, lysophospholipids and derivatives thereof; salts of alkylsulfates; salts of fatty acids; sodium docusate; acyl lactylates; mono-diacetylated tartaril acid esters of mono-diglycerides; succinylated monoglycerides; citric acid esters of mono-diglycerides; carnitines; and mixtures thereof.

More specifically, preferred ionic surfactants are lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholate, taurocholate, glycocholate, deoxycholate, taurodeoxycholate, chenodeoxycholate, glycodeoxycholate, glycochenodeoxycholate, taurochenodeoxycholate, ursodeoxycholate, tauroursodeoxycholate, glycoursodeoxycholate, cholylsarcosine, N-methyl taurocholate, caproate, caprylate, caprate, laurate, myristate, palmitate, oleate, ricinoleate, linoleate, linolenate, stearate, lauryl sulfate, teracecyl sulfate, docusate, lauroyl camitines, palmitoyl camitines, myristoyl carnitines, and salts and mixtures thereof.

Particularly preferred ionic surfactants are lecithin, lysolecithin, phosphatidylcholine, phosphatidylethanolamine, phosphatidylglycerol, lysophosphatidylcholine, PEG-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides cholate, taurocholate glycocholate, deoxycholate, taurodeoxycholate, glycodeoxycholate, cholylsarcosine, caproate, caprylate, caprate, laurate, oleate, lauryl sulfate, docusate, and salts and mixtures thereof, with the most preferred ionic surfactants being lecithin, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, taurocholate, caprylate, caprate, oleate, lauryl sulfate, docusate, and salts and mixtures thereof.

The carrier of the present compositions may include a combination of at least two surfactants, at least one of which is hydrophilic. In one embodiment, the present invention includes at two surfactants that are hydrophilic, and preferred hydrophilic surfactants are listed above. In another embodiment, the carrier includes at least one hydrophilic surfactant and at least one lipophilic surfactant. In this embodiment, preferred lipophilic surfactants are alcohols; polyoxyethylene alkylethers; fatty acids; glycerol fatty acid esters; acetylated glycerol fatty acid esters; lower alcohol fatty acids esters; polyethylene glycol fatty acids esters; polyethylene glycol glycerol fatty acid esters; polypropylene glycol fatty acid esters; polyoxyethylene glycerides; lactic acid derivatives of mono/diglycerides; propylene glycol diglycerides; sorbitan fatty acid esters; polyoxyethylene sorbitan fatty acid esters; polyoxyethylene-polyoxypropylene block copolymers; transesterified vegetable oils; sterols; sterol derivatives; sugar esters; sugar ethers; sucroglycerides; polyoxyethylene vegetable oils; and polyoxyethylene hydrogenated vegetable oils.

As with the hydrophilic surfactants, lipophilic surfactants can be reaction mixtures of polyols and fatty acids, glycerides, vegetable oils, hydrogenated vegetable oils, and sterols.

Preferably, the lipophilic surfactant is selected from the group consisting of fatty acids; lower alcohol fatty acid esters; polyethylene glycol glycerol fatty acid esters; polypropylene glycol fatty acid esters; polyoxyethylene glycerides; glycerol fatty acid esters; acetylated glycerol fatty acid esters; lactic acid derivatives of mono/diglycerides; sorbitan fatty acid esters; polyoxyethylene sorbitan fatty acid esters; polyoxyethylene-polyoxypropylene block copolymers; polyoxyethylene vegetable oils; polyoxyethylene hydrogenated vegetable oils; and reaction mixtures of polyols and fatty acids, glycerides, vegetable oils, hydrogenated vegetable oils, and sterols.

More preferred are lower alcohol fatty acids esters; polypropylene glycol fatty acid esters; propylene glycol fatty acid esters; glycerol fatty acid esters; acetylated glycerol fatty acid esters; lactic acid derivatives of mono/diglycerides; sorbitan fatty acid esters; polyoxyethylene vegetable oils; and mixtures thereof, with glycerol fatty acid esters and acetylated glycerol fatty acid esters being most preferred. Among the glycerol fatty acid esters, the esters are preferably mono- or diglycerides, or mixtures of mono- and diglycerides, where the fatty acid moiety is a C<6> to C<22> fatty acid. Also preferred are lipophilic surfactants which are the reaction mixture of polyols and fatty acids, glycerides, vegetable oils, hydrogenated vegetable oils, and sterols. Preferred polyols are polyethylene glycol, sorbitol, propylene glycol, and pentaerythritol.

Specifically preferred lipophilic surfactants include myristic acid; oleic acid; lauric acid; stearic acid; palmitic acid; PEG 1-4 stearate; PEG 2-4 oleate; PEG-4 dilaurate; PEG-4 dioleate; PEG-4 distearate; PEG-6 dioleate; PEG-6 distearate; PEG-8 dioleate; PEG 3-16 castor oil; PEG 5-10 hydrogenated castor oil; PEG 6-20 corn oil; PEG 6-20 almond oil; PEG-6 olive oil; PEG-6 peanut oil; PEG-6 palm kernel oil; PEG-6 hydrogenated palm kernel oil; PEG-4 capric/caprylic triglyceride, mono, di, tri, tetra esters of vegetable oil and sorbitol; pentaerythrityl di, tetra stearate, isostearate, oleate, caprylate, or caprate, polyglyceryl 2-4 oleate, stearate, or isostearate; polyglyceryl 4-10 pentaoleate; polyglyceryl-3 dioleate; polyglyceryl-6 dioleate; polyglyceryl-10 trioleate; polyglyceryl-3 distearate; propylene glycol mono- or diesters of a C<6> to C<20> fatty acid; monoglycerides of C<6> to C<20> fatty acids; acetylated monoglycerides of C<6> to C<20> fatty acids; diglycerides of C<6> to C<20> fatty acids; lactic acid derivatives of monoglycerides; lactic acid derivatives of diglycerides; cholesterol; phytosterol; PEG 5-20 soya sterol; PEG-6 sorbitan tetra, hexastearate; PEG-6 sorbitan tetraoleate; sorbitan monolaurate; sorbitan monopalmitate; sorbitan mono, trioleate; sorbitan mono, tristearate; sorbitan monoisostearate; sorbitan sesquioleate; sorbitan sesquistearate; PEG 2-5 oleyl ether; POE 2-4 lauryl ether; PEG-2 cetyl ether; PEG-2 stearyl ether; sucrose distearate; sucrose dipalmitate; ethyl oleate; isopropyl myristate; isopropyl palmitate; ethyl linoleate; isopropyl linoleate; and poloxamers.

Among the specifically preferred lipophilic surfactants, most preferred are oleic acid; lauric acid; glyceryl monocaprate; glyceryl monocaprylate; glyceryl monolaurate; glyceryl monooleate; glyceryl dicaprate; glyceryl dicaprylate; glyceryl dilaurate; glyceryl dioleate; acetylated monoglycerides; propylene glycol oleate; propylene glycol laurate; polyglyceryl-3 oleate; polyglyceryl-6 dioleate; PEG-6 corn oil; PEG-20 corn oil; PEG-20 almond oil; sorbitan monooleate; sorbitan monolaurate; POE-4 lauryl ether; POE-3 oleyl ether; ethyl oleate; and poloxamers.

3. Therapeutic Agents

As a general matter, the carrier used in the fill material of the present invention will have at least one therapeutic, or pharmaceutically active agent dissolved, disbursed, or otherwise incorporated therein. Any particular active agent may be administered in the form of a salt, ester, amide, prodrug, active metabolite, isomer, analog, fragment, or the like, provided that the salt, ester, amide, prodrug, active metabolite, isomer, analog or fragment, is pharmaceutically acceptable and pharmacologically active in the present context. Salts, esters, amides, prodrugs, metabolites, analogs, fragments, and other derivatives of the active agents may be prepared using standard procedures known to those skilled in the art of synthetic organic chemistry and described, for example, by J. March, Advanced Organic Chemistry: Reactions, Mechanisms and Structure, 4th Edition (New York: Wiley-Interscience, 1992).

For example, acid addition salts are prepared from a drug in the form of a free base using conventional methodology involving reaction of the free base with an acid. Suitable acids for preparing acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. An acid addition salt may be reconverted to the free base by treatment with a suitable base. Conversely, preparation of basic salts of acid moieties that may be present on an active agent may be carried out in a similar manner using a pharmaceutically acceptable base such as sodium hydroxide, potassium hydroxide, ammonium hydroxide, calcium hydroxide, trimethylamine, or the like. Preparation of esters involves transformation of a carboxylic acid group via a conventional esterification reaction involving nucleophilic attack of an RO⁻ moiety at the carbonyl carbon. Esterification may also be carried out by reaction of a hydroxyl group with an esterification reagent such as an acid chloride. Esters can be reconverted to the free acids, if desired, by using conventional hydrogenolysis or hydrolysis procedures. Amides may be prepared from esters, using suitable amine reactants, or they may be prepared from an anhydride or an acid chloride by reaction with ammonia or a lower alkyl amine. Prodrugs and active metabolites may also be prepared using techniques known to those skilled in the art or described in the pertinent literature. prodrugs are typically prepared by covalent attachment of a moiety that results in a compound that is therapeutically inactive until modified by an individual's metabolic system.

Other derivatives and analogs of the active agents may be prepared using standard techniques known to those skilled in the art of synthetic organic chemistry, or may be deduced by reference to the pertinent literature. In addition, chiral active agents may be in isomerically pure form, or they may be administered as a racemic mixture of isomers.

The pharmaceutically active agent is dissolved or disbursed (i.e. suspended) in the fill material. No particular limitation is placed on the specific pharmaceutically active agent that can be included. Rather, the carrier materials recited herein are capable of solubilizing or suspending, and delivering a wide variety of therapeutic agents. The therapeutic agents can be hydrophilic, amphiphilic, or lipophilic. Optionally, the therapeutic agent can be present in a first, solubilized amount, and a second, non-solubilized (suspended) amount. Such therapeutic agents can be any agents having therapeutic or other value when administered to an animal, particularly to a mammal, such as drugs, nutrients, and cosmetics (cosmeceuticals). It should be understood that while the invention is described with particular reference to its value in the form of aqueous dispersions, the invention is not so limited. Thus, drugs, diagnostics, nutrients or cosmetics which derive their therapeutic or other value from, for example, topical or transdermal administration, are still considered to be suitable for use in the present invention.

A wide variety of active agents may be administered using the dosage forms of the present invention. No limitation is perceived thereon, except to the extent that a particular active agent prevents or hinders the functioning of the present dosage forms to the extent that they become unsuitable for use. However, as the dosage forms of the present invention allow a significant latitude for adjustment, it is expected that attunement of one or more specific parameters will be sufficient to accommodate virtually any active agent desired to be delivered. Examples of active agent contemplated for administration with the dosage forms of the present invention include without limitation various classes of active agents such as, analgesic agents, anesthetic agents, anti-anginal agents, antiarthritic agents, anti-arrhythmic agents, antiasthmatic agents, antibacterial agents, anti-BPH agents, anticancer agents, anticholinergic agents, anticoagulants, anticonvulsants, antidepressants, antidiabetic agents, antidiarrheals, anti-epileptic agents, antifungal agents, antigout agents, antihelminthic agents, antihistamines, antihypertensive agents, antiinflammatory agents, antimalarial agents, antimigraine agents, antimuscarinic agents, antinauseants, antineoplastic agents, anti-obesity agents, antiosteoporosis agents, antiparkinsonism agents, antiprotozoal agents, antipruritics, antipsychotic agents, antipyretics, antispasmodics, antithyroid agents, antitubercular agents, antiulcer agents, anti-urinary incontinence agents, antiviral agents, anxiolytics, appetite suppressants, attention deficit disorder (ADD) and attention deficit hyperactivity disorder (ADHD) drugs, calcium channel blockers, cardiac inotropic agents, beta-blockers, central nervous system stimulants, cognition enhancers, corticosteroids, COX-2 inhibitors, decongestants, diuretics, gastrointestinal agents, genetic materials, histamine receptor antagonists, hormonolytics, hypnotics, hypoglycemic agents, immunosuppressants, keratolytics, leukotriene inhibitors, lipid-regulating agents, macrolides, mitotic inhibitors, muscle relaxants, narcotic antagonists, neuroleptic agents, nicotine, nutritional oils, parasympatholytic agents, sedatives, sex hormones, sympathomimetic agents, tranquilizers, vasodilators, vitamins, and combinations thereof. Active agents that may be administered according to the invention also include nutrients, cosmeceuticals, diagnostic agents, and nutritional agents. Some agents, as will be appreciated by those of ordinary skill in the art, and as may be deduced from the discussion below, are encompassed by two or more of the aforementioned groups or other uses that may be found appropriate.

Among the various active agent categories, preferred classes of active agents for administration using the present method and formulations are lipid regulating agents, sex hormones, anti-hypertensive agents, anti-diabetic agents, anti-viral agents (including protease inhibitors), gastrointestinal agents, agents for treating neurodegenerative diseases (including anti-parkinson's and anti-Alzheimer's), anxiolytics, sedatives, hypnotics, agents for treating headaches (including anti-migraine agents), neuroleptic drugs (including anti-depressants, anti-manics, anti-psychotics) and combinations of any of the foregoing:

Lipid-regulating agents that are generally classified as hydrophobic include HMG CoA reductase inhibitors such as atorvastatin, simvastatin, fluvastatin, pravastatin, lovastatin, cerivastatin, rosuvastatin, and pitavastatin, as well as other lipid-lowering (“antihyperlipidemic”) agents such as bezafibrate, beclobrate, binifibrate, ciprofibrate, clinofibrate, clofibrate, clofibric acid, ezetimibe, etofibrate, fenofibrate, fenofibric acid, gemfibrozil, lifibrol, nicofibrate, pirifibrate, probucol, ronifibrate, simfibrate, and theofibrate. A particularly preferred lipid-regulating agent that may be administered using the methods and formulations of the invention is fenofibrate.

Sex hormones that are preferred for administration according to the invention include progestins (progestogens), estrogens, and combinations thereof. Progestins include acetoxypregnenolone, allylestrenol, anagestone acetate, chlormadinone acetate, cyproterone, cyproterone acetate, desogestrel, dihydrogesterone, dimethisterone, ethisterone (17α-ethinyltestosterone), ethynodiol diacetate, flurogestone acetate, gestadene, hydroxyprogesterone, hydroxyprogesterone acetate, hydroxyprogesterone caproate, hydroxymethylprogesterone, hydroxymethylprogesterone acetate, 3-ketodesogestrel, levonorgestrel, lynestrenol, medrogestone, medroxyprogesterone acetate, megestrol, megestrol acetate, melengestrol acetate, norethindrone, norethindrone acetate, norethisterone, norethisterone acetate, norethynodrel, norgestimate, norgestrel, norgestrienone, normethisterone, progesterone, and trimgestone. Also included within this general class are estrogens, e.g.: estradiol (i.e., 1,3,5-estratriene-3,17β-diol, or “17β-estradiol”) and its esters, including estradiol benzoate, valerate, cypionate, heptanoate, decanoate, acetate and diacetate; 17α-estradiol; ethinylestradiol (i.e., 17α-ethinylestradiol) and esters and ethers thereof, including ethinylestradiol 3-acetate and ethinylestradiol 3-benzoate; estriol and estriol succinate; polyestrol phosphate; estrone and its esters and derivatives, including estrone acetate, estrone sulfate, and piperazine estrone sulfate; quinestrol; mestranol; and conjugated equine estrogens. In many contexts, e.g., in female contraception and in hormone replacement therapy (HRT), a combination of a progestin and estrogen is used, e.g., progesterone and 17 β-estradiol. For HRT, an androgenic agent may be advantageously included as well. Androgenic agents for this purpose include, for example, dehydroepiandrosterone (DHEA; also termed “prasterone”), sodium dehydroepiandrosterone sulfate, 4-dihydrotestosterone (DHT; also termed “stanolone”), and testosterone, and pharmaceutically acceptable esters of testosterone and 4-dihydrotestosterone, typically esters formed from the hydroxyl group present at the C-17 position, including, but not limited to, the enanthate, propionate, cypionate, phenylacetate, acetate, isobutyrate, buciclate, heptanoate, decanoate, undecanoate, caprate and isocaprate esters.

Androgenic agents may also be administered for other purposes well known in the art. In addition to the androgenic agents enumerated above, other androgenic agents include, but are not limited to, androsterone, androsterone acetate, androsterone propionate, androsterone benzoate, androstenediol, androstenediol-3-acetate, androstenediol-17-acetate, androstenediol-3, 17-diacetate, androstenediol-17-benzoate, androstenediol-3-acetate-17-benzoate, androstenedione, ethylestrenol, oxandrolone, nandrolone phenpropionate, nandrolone decanoate, nandrolone furylpropionate, nandrolone cyclohexane-propionate, nandrolone benzoate, nandrolone cyclohexanecarboxylate, stanozolol, dromostanolone, and dromostanolone propionate.

Antihypertensive agents include, without limitation, amlodipine, benazepril, benidipine, candesartan, captopril, carvedilol, darodipine, dilitazem, diazoxide, doxazosin, enalapril, epleronone, eposartan, felodipine, fenoldopam, fosinopril, guanabenz, iloprost, imidapril, irbesartan, isradipine, lercardinipine, lisinopril, losartan, mibefradil, minoxidil, nebivolol, nicardipine, nifedipine, nimodipine, nisoldipine, olmesartan, omapatrilat, phenoxybenzamine, pindolol, prazosin, quinapril, reserpine, semotiadil, sitaxsentan, terazosin, telmisartan, trandolapril, and valsartan.

Anti-diabetic agents include, by way of example, acetohexamide, chlorpropamide, ciglitazone, farglitazar, glibenclamide, gliclazide, glipizide, glucagon, glyburide, glymepiride, miglitol, pioglitazone, nateglinide, pimagedine, repaglinide, rosiglitazone, tolazamide, tolbutamide, triampterine, and troglitazone.

Antiviral agents that can be delivered using the present methods and dosage forms include the antiherpes agents acyclovir, famciclovir, foscamet, ganciclovir, idoxuridine, sorivudine, trifluridine, valacyclovir, and vidarabine, and other antiviral agents such as abacavir, amantadine, amprenavir, cidofovir, delviridine, didanosine, efavirenz, indinavir, interferon alpha, lamivudine, lobucavir, lopinavir, nelfinavir, nevirapine, oseltamivir, ribavirin, rimantadine, ritonavir, saquinavir, stavudine, tipranavir, valganciclovir, zanamivir, zalcitabine, and zidovudine; and other antiviral agents such as abacavir, indinavir, interferon alpha, nelfmavir, ribavirin, rimantadine, tipranavir, ursodeoxycholic acid, and valganciclovir.

Gastrointestinal agnts, such as alosetron, basalazide, bisacodyl, budesonide, cilansetron, cimetidine, cisapride, diphenoxylate, domperidone, esomeprazole, famotidine, granisetron, lafutidine, lansoprazole, leminoprazole, loperamide, merropenum, mesalazine, mesalamine, nitisonone, nizatidine, olsalazine, omeprazole, ondansetron, pantoprazole, palonosetron, pariprazole, rabeprazole sodium, ransoprazole, ranitidine, risperidone, sulphasalazine, and tegaserod;

Neuroleptic drugs, including antidepressant drugs, antimanic drugs, and antipsychotic agents, wherein antidepressant drugs include (a) the tricyclic antidepressants such as amoxapine, amitriptyline, clomipramine, desipramine, doxepin, imipramine, maprotiline, nortriptyline, protriptyline, and trimipramine, (b) the serotonin reuptake inhibitors citalopram, escitalopram, fluoxetine, fluvoxamine, paroxetine, sertraline, and venlafaxine, (c) monoamine oxidase inhibitors such as phenelzine, tranylcypromine, and (−)-selegiline, and (d) other antidepressants such as aprepitant, bupropion, duloxetine, gepirone, igmesine, lamotrigine, maprotiline, mianserin, mirtazapine, nefazodone, rabalzotan, sunepitron, trazodone and venlafaxine, and wherein antimanic and antipsychotic agents include (a) phenothiazines such as acetophenazine, acetophenazine maleate, chlorpromazine, chlorpromazine hydrochloride, fluphenazine, fluphenazine hydrochloride, fluphenazine enanthate, fluphenazine decanoate, mesoridazine, mesoridazine besylate, perphenazine, thioridazine, thioridazine hydrochloride, trifluoperazine, and trifluoperazine hydrochloride, (b) thioxanthenes such as chlorprothixene, thiothixene, and thiothixene hydrochloride, and (c) other heterocyclic drugs such as carbamazepine, clozapine, droperidol, haloperidol, haloperidol decanoate, loxapine succinate, molindone, molindone hydrochloride, olanzapine, perospirone, pimozide, quetiapine, risperidone, sertindole, and ziprasidone.

Agents for treating headaches, including anti-migraine agents, such as almotriptan, butorphanol, dihydroergotamine, dihydroergotamine mesylate, eletriptan, ergotamine, frovatriptan, methysergide, naratriptan, pizotyline, rizatriptan, sumatriptan, tonaberstat, and zolmitriptan;

Agents to treat neurodegenerative diseases, including active agents for treating Alzheimer's disease such as akatinol, donezepil, donepezil hydrochloride, dronabinol, galantamine, ipidracine, neotrofin, rasagiline, physostigmine, physostigmine salicylate, propentoffyline, quetiapine, rivastigmine, tacrine, tacrine hydrochloride, thalidomide, and xaliproden; active agents for treating Huntington's Disease, such as fluoxetine and carbamazepine; anti-parkinsonism drugs useful herein include amantadine, apomorphine, bromocriptine, entacapone, levodopa (particularly a levodopa/carbidopa combination), lysuride, pergolide, pramipexole, rasagiline, riluzole, ropinirole, selegiline, sumanirole, tolcapone, trihexyphenidyl, and trihexyphenidyl hydrochloride; and active agents for treating ALS such as the anti-spastic agents baclofen, diazemine, riluzole, and tizanidine; and active agents for multiple sclerosis such as glatiramer.

Anxiolytics sedatives, and hypnotics, such as alprazolam, amylobarbitone, barbitone, bentazepam, bromazepam, bromperidol, brotizolam, butobarbitone, carbromal, chlordiazepoxide, chlormethiazole, chlorpromazine, chlorprothixene, clonazepam, clobazam, clotiazepam, clozapine, dexmethylphenidate (d-threomethylphenidate) diazepam, droperidol, ethinamate, flunanisone, flunitrazepam, triflupromazine, flupenthixol decanoate, fluphenazine, flurazepam, gabapentin, gaboxadol, γ-hydroxybutyrate, haloperidol, lamotrigine, lorazepam, lormetazepam, medazepam, meprobamate, mesoridazine, methaqualone, methylphenidate, midazolam, modafinil, molindone, nitrazepam, olanzapine, oxazepam, pentobarbitone, perphenazine pimozide, pregabalin, prochlorperazine, pseudoephedrine, quetiapine, rispiridone, rohypnol, sertindole, siramesine, sulpiride, sunepitron, temazepam, thioridazine, triazolam, zaleplon, zolpidem, and zopiclone;

Other therapeutic agents that can be delivered using the present methods and formulations include the following representative compounds:

Anti-inflammatory agents and non-opioid analgesics, such as aloxiprin, amiprilose, auranofin, azapropazone, azathioprine, benorylate, boswellic acid, butorphenol, capsaicin, celecoxib, diclofenac, diflunisal, esonarimod, etodolac, fenbufen, fenoprofen calcium, flurbiprofen, ibuprofen, indomethacin, ketoprofen, ketorolac, leflunomide, meclofenamic acid, mefenamic acid, nabumetone, naproxen, novantrone, oxaprozin, oxyphenbutazone, parecoxib, phenylbutazone, piclamilast, piroxicam, rofecoxib, ropivacaine, sulindac, tetrahydrocannabinol, tramadol, tromethamine, valdecoxib, and ziconotide, as well as the urinary analgesics phenazopyridine and tolterodine;

Anti-angina agents, such as mibefradil, refludan, nalmefene, carvedilol, cromafiban, lamifiban, fasudil, ranolazine, tedisamil, nisoldipine, and tizanidine;

Antihelminthics, such as albendazole, bephenium hydroxynaphthoate, cambendazole, dichlorophen, ivermectin, mebendazole, oxamniquine, oxfendazole, oxantel embonate, praziquantel, pyrantel embonate and thiabendazole;

Anti-arrhythmic agents, such as amiodarone, disopyramide, flecainide acetate and quinidine sulfate;

Anti-asthma agents, such as fudosteine, zileuton, zafirlukast, terbutaline sulfate, montelukast, pranlukast, levalbuterol, ramatroban, suplatast, and albuterol;

Anti-bacterial agents, such as alatrofloxacin, azithromycin, baclofen, benethamine penicillin, cinoxacin, ciprofloxacin, cefoselis, ceftibuten, clarithromycin, clofazimine, cloxacillin, dalfopristine, demeclocycline, dirithromycin, doxycycline, ecenofloxacin, erythromycin, ethionamide, furazolidone, grepafloxacin, imipenem, levofloxacin, linezolid, lorefloxacin, moxifloxacin, nalidixic acid, nitrofurantoin, norfloxacin, ofloxacin, quinupritin, rifampicin, rifabutine, rifapentine, ritipenem, sparfloxacin, spiramycin, sulphabenzamide, sulphadoxine, sulphamerazine, sulphacetamide, sulphadiazine, sulphafurazole, sulphamethoxazole, sulphapyridine, tazobactum, tetracycline, tosufloxacin, trimethoprim, trovafloxacin, and vancomycin;

Anti-cancer agents and immunosuppressants, such as alitretinoin, aminoglutethimide, amsacrine, anastrozole, azathioprine, bexarotene, bicalutamide, biricodar, bisantrene, busulfan, camptothecin, candoxatril, capecitabine, cisplatin, cytarabine, chlorambucil, cyclosporin, dacarbazine, decitabine, ellipticine, estramustine, etoposide, examorelin, examestane, fludarabine, gemcitabine, imatinib, irinotecan, lasofoxifene, letrozole, lomustine, melphalan, mercaptopurine, methotrexate, mitomycin, mitotane, mitoxantrone, mofetil, mycophenolate, nebivolol, nilutamide, oxaliplatin, paclitaxel, palonosetron, procarbazine, ramipril, rubitecan, sirolimus, tacrolimus, tamoxifen, teniposide, testolactone, thalidomide, tirapazamine, topotecan, toremifene citrate, vitamin A, vitamin A derivatives, venorelbine, and zacopride;

Anti-coagulants and other agents for preventing and treating stroke, such agatroban, cilostazol, citicoline, clopidogrel, cromafiban, dexanabinol, dicumarol, dipyridamole, nicoumalone, oprelvekin, ozagrel, perindopril erbumine, phenindione, ramipril, repinotan, ticlopidine, tirofiban, and heparin, including heparin salts formed with organic or inorganic bases, and low molecular weight heparin, i.e., heparin fragments generally having a weight average molecular weight in the range of about 1000 to about 10,000 D and exemplified by enoxaparin, dalteparin, danaproid, gammaparin, nadroparin, ardeparin, tinzaparin, certoparin, and reviparin;

Anti-diabetics, such as acetohexamide, chlorpropamide, farglitazar, glibenclamide, gliclazide, glipizide, glimepiride, miglitol, nateglinide, pimagedine, pioglitazone, repaglinide, rosiglitazone, tolazamide, tolbutamide, troglitazone, and voglibose;

Anti-epileptics, such as beclamide, carbamazepine, carbatrol, clobazam, clonazepam, divalproex sodium, ethotoin, felbamate, fosphenytoin, levetriacetam, lamotrigine, methoin, methsuximide, methylphenobarbitone, oxcarbazepine, paramethadione, phenacemide, phenobarbitone, phenytoin, phensuximide, primidone, sulthiame, tiagabine, tolcapone, topiramate, valproic acid, vigabatrin, and zonisamide;

Anti-fungal agents, such as anidulafungin, amphotericin, butenafine, butoconazole nitrate, clotrimazole, econazole nitrate, fluconazole, flucytosine, griseofulvin, itraconazole, ketoconazole, liranaftate, miconazole, natamycin, nystatin, sulconazole nitrate, oxiconazole, terbinafine, terconazole, tioconazole and undecenoic acid;

Anti-gout agents, such as allopurinol, probenecid and sulphin-pyrazone;

Antihistamines and allergy medications, such as acrivastine, astemizole, chlorpheniramine, cinnarizine, cetirizine, clemastine, cyclizine, cyproheptadine, desloratadine, dexchlorpheniramine, dimenhydrinate, diphenhydramine, epinastine, fexofenadine, flunarizine, loratadine, meclizine, mizolastine, oxatomide, and terfenadine;

Anti-malarials, such as amodiaquine, chloroquine, chlorproguanil, halofantrine, mefloquine, proguanil, pyrimethamine and quinine sulfate;

Anti-muscarinic agents, such as atropine, benzhexol, biperiden, ethopropazine, hyoscyamine, mepenzolate bromide, oxyphencyclimine, scopolamine, and tropicamide;

Anti-protozoal agents, such as atovaquone, benznidazole, clioquinol, decoquinate, diiodohydroxyquinoline, diloxanide furoate, dinitolmide, furazolidone, metronidazole, nimorazole, nitrofurazone, omidazole and tinidazole;

Anti-thyroid agents, such as carbimazole, paricalcitol, and propylthiouracil;

Anti-tussives, such as benzonatate;

Appetite suppressants, anti-obesitv drugs and drugs for treatment of eating disorders, such as amphetamine, bromocriptine, dextroamphetamine, diethylpropion, gherelin, lintitript, mazindol, methamphetamine, orlistat, phentermine, and topiramate;

Cardiovascular drugs, including: angiotensin converting enzyme (ACE) inhibitors such as enalapril, ramipril, perindopril erbumine, 1-carboxymethyl-3-1-carboxy-3-phenyl-(1S)-propylamino-2,3,4,5-tetrahydro-1H-(3S)-11-benzazepine-2-one, 3-(5-amino-1-carboxy-1S-pentyl)amino-2,3,4,5-tetrahydro-2-oxo-3 S-1H-1-benzazepine-1-acetic acid or 3-(1-ethoxycarbonyl-3-phenyl-(1S)-propylamino)-2,3,4,5-tetrahydro-2-oxo-(3S)-benzazepine-1-acetic acid monohydrochloride; cardiac glycosides and cardiac inotropes such as amrinone, digoxin, digitoxin, enoximone, lanatoside C, medigoxin, and milrinone; calcium channel blockers such as verapamil, nifedipine, nicardipene, felodipine, isradipine, nimodipine, amlodipine and diltiazem; beta-blockers such as acebutolol, alprenolol, atenolol, labetalol, metoprolol, nadolol, oxyprenolol, pindolol, propafenone, propranolol, esmolol, sotalol, timolol, and acebutolol; antiarrhythmics such as mexiletene, moricizine, dofetilide, ibutilide, nesiritide, procainamide, quinidine, disopyramide, lidocaine, phenytoin, tocainide, mexiletine, flecainide, encainide, bretylium and amiodarone; cardioprotective agents such as dexrazoxane and leucovorin; vasodilators such as nitroglycerin; diuretic agents such as azetazolamide, amiloride, bendroflumethiazide, bumetanide, chlorothiazide, chlorthalidone, ethacrynic acid, furosemide, hydrochlorothiazide, metolazone, nesiritide, spironolactone, and triamterine; and miscellaneous cardiovascular drugs such as dopradil, midodrine, monatepil, monteplase, nexopamil, ranolazine, and pilsicainide;

Corticosteroids, such as beclomethasone, betamethasone, budesonide, cortisone, desoxymethasone, dexamethasone, fludrocortisone, flunisolide, fluocortolone, fluticasone propionate, hydrocortisone, methylprednisolone, prednisolone, prednisone and triamcinolone;

Cytoprotectant/Antioxidant, such as dosmalfate, curcumin, edavarone;

Erectile dysfunction drugs, such as apomorphine, phentolamine, and vardenafil;

Keratolytics such as such as acetretin, calcipotriene, calcifediol, calcitriol, cholecalciferol, ergocalciferol, etretinate, retinoids, targretin, and tazarotene;

Muscle relaxants, such as cyclobenzaprine, dantrolene sodium, mexilitene, and tizanidine HCl;

Nitrates and other anti-anginal agents, such as amyl nitrate, glyceryl trinitrate, isosorbide dinitrate, isosorbide mononitrate and pentaerythritol tetranitrate;

Nutritional agents, such as calcitriol, carotenes, dihydrotachysterol, essential fatty acids, non-essential fatty acids, phytonadiol, vitamin A, vitamin B₂, vitamin D, vitamin E and vitamin K.

Opioid analgesics, such as alfentanil, apomorphine, buprenorphine, butorphanol, codeine, dextropropoxyphene, diamorphine, dihydrocodeine, fentanyl, hydrocodone, hydromorphone, levorphanol, meperidine, meptazinol, methadone, morphine, nalbuphine, oxycodone, oxymorphone, pentazocine, propoxyphene, sufentanil, and tramadol;

Stimulants, including active agents for treating narcolepsy, attention deficit disorder (ADD) and attention deficit hyperactivity disorder (ADHD), such as amphetamine, dexamphetamine, dexfenfluramine, fenfluramine, mazindol, methylphenidate (including d-threo-methylphenidate, or “dexmethylphenidate,” as well as racemic d,1-threo-methylphenidate), modafinil, pemoline, and sibutramine.

Peptidyl drugs include therapeutic peptides and proteins per se, whether naturally occurring, chemically synthesized, recombinantly produced, and/or produced by biochemical (e.g., enzymatic) fragmentation of larger molecules, and may contain the native sequence or an active fragment thereof. Specific peptidyl drugs include, without limitation, the peptidyl hormones activin, amylin, angiotensin, atrial natriuretic peptide (ANP), calcitonin, calcitonin gene-related peptide, calcitonin N-terminal flanking peptide, ciliary neurotrophic factor (CNTF), corticotropin (adrenocorticotropin hormone, ACTH), corticotropin-releasing factor (CRF or CRH), epidermal growth factor (EGF), follicle-stimulating hormone (FSH), gastrin, gastrin inhibitory peptide (GIP), gastrin-releasing peptide, gonadotropin-releasing factor (GnRF or GNRH), growth hormone releasing factor (GRF, GRH), human chorionic gonadotropin (hCH), inhibin A, inhibin B, insulin, luteinizing hormone (LH), luteinizing hormone-releasing hormone (LHRH), α-melanocyte-stimulating hormone, β-melanocyte-stimulating hormone, γ-melanocyte-stimulating hormone, melatonin, motilin, oxytocin (pitocin), pancreatic polypeptide, parathyroid hormone (PTH), placental lactogen, prolactin (PRL), prolactin-release inhibiting factor (PIF), prolactin-releasing factor (PRF), secretin, somatotropin (growth hormone, GH), somatostatin (SIF, growth hormone-release inhibiting factor, GIF), thyrotropin (thyroid-stimulating hormone, TSH), thyrotropin-releasing factor (TRH or TRF), thyroxine, vasoactive intestinal peptide (VIP),and vasopressin. Other peptidyl drugs are the cytokines, e.g., colony stimulating factor 4, heparin binding neurotrophic factor (HBNF), interferon-α, interferon α-2a, interferon α-2b, interferon α-n3, interferon -β, etc., interleukin-1, interleukin-2, interleukin-3, interleukin-4, interleukin-5, interleukin-6, etc., tumor necrosis factor, tumor necrosis factor-α, granuloycte colony-stimulating factor (G-CSF), granulocyte-macrophage

colony-stimulating factor (GM-CSF), macrophage colony-stimulating factor, midkine (MD), and thymopoietin. Still other peptidyl drugs that can be advantageously delivered using the methodology and formulations of the present invention include endorphins (e.g., dermorphin, dynorphin, α-endorphin, β-endorphin, γ-endorphin, σ-endorphin, [Leu⁵]enkephalin, [Met⁵]enkephalin, substance P), kinins (e.g., bradykinin, potentiator B, bradykinin potentiator C, kallidin), LHRH analogues (e.g., buserelin, deslorelin, fertirelin, goserelin, histrelin, leuprolide, lutrelin, nafarelin, tryptorelin), and the coagulation factors, such as α₁-antitrypsin, α₂-macroglobulin, antithrombin III, factor I (fibrinogen), factor II (prothrombin), factor III (tissue prothrombin), factor V (proaccelerin), factor VII (proconvertin), factor VIII (antihemophilic globulin or AHG), factor IX (Christmas factor, plasma thromboplastin component or PTC), factor X (Stuart-Power factor), factor XI (plasma thromboplastin antecedent or PTA), factor XII (Hageman factor), heparin cofactor II, kallikrein, plasmin, plasminogen, prekallikrein, protein C, protein S, and thrombomodulin and combinations thereof.

Genetic material may also be delivered using the present methods and formulations, including, for example, nucleic acids, RNA, DNA, recombinant RNA, recombinant DNA, antisense RNA, antisense DNA, ribozymes, ribooligonucleotides, deoxyribonucleotides, antisense ribooligonucleotides, and antisense deoxyribooligonucleotides. Representative genes include those encoding for vascular endothelial growth factor, fibroblast growth factor, Bcl-2, cystic fibrosis transmembrane regulator, nerve growth factor, human growth factor, erythropoietin, tumor necrosis factor, and interleukin-2, as well as histocompatibility genes such as HLA-B7.

Other actives: dutasetride for hair loss, granelix acetate for female infertility, incadronic acid for cancer or osteoporosis, pergolide for dopamine agonist activity, ritapentine, perenzepine, telenzepine, titanicene, limaprost, olopatidine, falecalcitriol, caldiribine, piapenum, farapenum, piracetam, tianeptine, adrafinil, vinpocetine, idebenone, oxiracetam, aniracetam, ketamine, ertapenum, cabergoline, acamprostate, nevibulol;

The active agent of the present invention can be hydrophobic, amphiphilic, or hydrophilic. The intrinsic water solubility of those active agents referred to as “hydrophobic” herein, i.e., the aqueous solubility of the active agent in electronically neutral, non-ionized form, is generally less than 1% by weight, and typically less than 0.1% or 0.01% by weight. Hydrophilic and amphiphilic active agents herein (which, unless otherwise indicated, are collectively referred to herein as “hydrophilic” active agents) have apparent water solubilities of at least 0.1% by weight, and typically at least 1% by weight. Both hydrophobic active agents and hydrophilic active agents may be selected from any of the active agent classes enumerated earlier in this section.

Further, it should be appreciated that the categorization of an active ingredient as hydrophobic or hydrophilic may change, depending upon the particular salts, isomers, analogs and derivatives used. For example, certain active agents indicated as hydrophobic may be readily converted to and commercially available in hydrophilic form, e.g., by ionizing a non-ionized active agent so as to form a pharmaceutically acceptable, pharmacologically active salt. Conversely, certain active agents indicated as hydrophilic may be readily converted to and commercially available in hydrophobic form, e.g., by neutralization, esterification, or the like. Thus, it should be understood that the above categorization of certain active agents as hydrophilic or hydrophobic is not intended to be limiting.

Specific, non-limiting examples of suitable hydrophobic active ingredients are: acetretin, acetyl coenzyme Q, albendazole, albuterol, aminoglutethimide, amiodarone, amlodipine, amphetamine, amphotericin B, atorvastatin, atovaquone, azithromycin, baclofen, beclomethasone, benezepril, benzonatate, betamethasone, bicalutanide, budesonide, bupropion, busulfan, butenafine, calcifediol, calcipotriene, calcitriol, camptothecin, candesartan, capsaicin, carbamezepine, carotenes, celecoxib, cerivastatin, cetirizine, chlorpheniramine, cholecalciferol, cilostazol, cimetidine, cinnarizine, ciprofloxacin, cisapride, clarithromycin, clemastine, clomiphene, clomipramine, clopidogrel, codeine, coenzyme Q10, cyclobenzaprine, cyclosporin, danazol, dantrolene, dexchlorpheniramine, diclofenac, dicoumarol, digoxin, dehydroepiandrosterone, dihydroergotamine, dihydrotachysterol, dirithromycin, donezepil, efavirenz, eposartan, ergocalciferol, ergotamine, essential fatty acid sources, esomeprazole, estradiol, etodolac, etoposide, famotidine, fenofibrate, fentanyl, fexofenadine, finasteride, fluconazole, flurbiprofen, fluvastatin, fosphenytoin, frovatriptan, furazolidone, gabapentin, gemfibrozil, glibenclamide, glipizide, glyburide, glimepiride, griseofulvin, halofantrine, ibuprofen, irbesartan, irinotecan, isosorbide dinitrate, isotretinoin, itraconazole, ivermectin, ketoconazole, ketorolac, lamotrigine, lansoprazole, leflunomide, lisinopril, loperamide, loratadine, lovastatin, L-thryroxine, lutein, lycopene, medroxyprogesterone, mifepristone, mefloquine, megestrol acetate, methadone, methoxsalen, metronidazole, miconazole, midazolam, miglitol, minoxidil, mitoxantrone, montelukast, nabumetone, nalbuphine, naratriptan, nelfinavir, nifedipine, nisoldipine, nilutanide, nitrofurantoin, nizatidine, omeprazole, oprevelkin, oxaprozin, paclitaxel, pantoprazole, paracalcitol, paroxetine, pentazocine, pioglitazone, pizofetin, pravastatin, prednisolone, probucol, progesterone, pseudoephedrine, pyridostigmine, rabeprazole, raloxifene, repaglinide, rifabutine, rifapentine, rimexolone, ritanovir, rizatriptan, rofecoxib, rosiglitazone, saquinavir, sertraline, sibutramine, sildenafil citrate, simvastatin, sirolimus, spironolactone, sumatriptan, tacrine, tacrolimus, tamoxifen, tamsulosin, targretin, tazarotene, telmisartan, teniposide, terbinafine, terazosin, tetrahydrocannabinol, tiagabine, ticlopidine, tirofibran, tizanidine, topiramate, topotecan, toremifene, tramadol, tretinoin, troglitazone, trovafloxacin, ubidecarenone, valsartan, venlafaxine, verteporfin, vigabatrin, vitamin A, vitamin D, vitamin E, vitamin K, zafirlukast, zileuton, ziprasidone, zolmitriptan, zolpidem, and zopiclone. Of course, salts, isomers and derivatives of the above-listed hydrophobic active ingredients may also be used, as well as mixtures therof.

Specific, non-limiting examples of suitable hydrophilic active ingredients include: acarbose; acyclovir; acetyl cysteine; acetylcholine chloride; alatrofloxacin; alendronate; alglucerase; amantadine hydrochloride; ambenomium; amifostine; amiloride hydrochloride; aminocaproic acid; amphotericin B; antihemophilic factor (human); antihemophilic factor (porcine); antihemophilic factor (recombinant); aprotinin; asparaginase; atenolol; atracurium besylate; atropine; azithromycin; aztreonam; BCG vaccine; bacitracin; becalermin; belladona; bepridil hydrochloride; bleomycin sulfate; calcitonin human; calcitonin salmon; carboplatin; capecitabine; capreomycin sulfate; cefamandole nafate; cefazolin sodium; cefepime hydrochloride; cefixime; cefonicid sodium; cefoperazone; cefotetan disodium; cefotaxime; cefoxitin sodium; ceftizoxime; ceftriaxone; cefuroxime axetil; cephalexin; cephapirin sodium; cholera vaccine; chorionic gonadotropin; cidofovir; cisplatin; cladribine; clidinium bromide; clindamycin and clindamycin derivatives; ciprofloxacin; clodronate; colistimethate sodium; colistin sulfate; corticotropin; cosyntropin; coromlyn sodium; cytarabine; dalteparin sodium; danaparoid; desferrioxamine; denileukin diftitox; desmopressin; diatrizoate meglumine and diatrizoate sodium; dicyclomine; didanosine; dirithromycin; dopamine hydrochloride; dornase alpha; doxacurium chloride; doxorubicin; etidronate disodium; enalaprilat; enkephalin; enoxaparin; enoxaparin sodium; ephedrine; epinephrine; epoetin alpha; erythromycin; esmolol hydrochloride; factor IX; famciclovir; fludarabine; fluoxetine; foscarnet sodium; ganciclovir; granulocyte colony stimulating factor; granulocyte-macrophage stimulating factor; recombinant human growth hormones; bovine growth hormone; gentamycin; glucagon; glycopyrolate; gonadotropin releasing hormone and synthetic analogs thereof; GnRH; gonadorelin; grepafloxacin; haemophilus B conjugate vaccine; Hepatitis A virus vaccine inactivated; Hepatitis B virus vaccine inactivated; heparin sodium; indinavir sulfate; influenza virus vaccine; interleukin-2; interleukin-3; insulin-human; insulin lispro; insulin procine; insulin NPH; insulin aspart; insulin glargine; insulin detemir; interferon alpha; interferon beta; ipratropium bromide; ifosfamide; Japanese encephalitis virus vaccine; lamivudine; leucovorin calcium; leuprolide acetate; levofloxacin; lincomycin and lincomycin derivatives; lobucavir; lomefloxacin; loracarbef; mannitol; measles virus vaccine; meningococcal vaccine; menotropins; mepenzolate bromide; mesalamine; methenamine; methotrexate; methscopolamine; metformin hydrochloride; metoprolol; mezocillin sodium; mivacurium chloride; mumps viral vaccine; nedocromil sodium; neostigmine bromide; neostigmine methyl sulfate; neurontin; norfloxacin; octreotide acetate; ofloxacin; olpadronate; oxytocin; pamidronate disodium; pancuronium bromide; paroxetine; perfloxacin; pentamidine isethionate; pentostatin; pentoxifylline; periciclovir; pentagastrin; phentolamine mesylate; phenylalanine; physostigmine salicylate; plague vaccine; piperacillin sodium; platelet derived growth factor; pneumococcal vaccine polyvalent; poliovirus vaccine (inactivated); poliovirus vaccine live (OPV); polymyxin B sulfate; pralidoxime chloride; pramlintide; pregabalin; propafenone; propantheline bromide; pyridostigmine bromide; rabies vaccine; residronate; ribavarin; rimantadine hydrochloride; rotavirus vaccine; salmeterol xinafoate; sincalide; small pox vaccine; solatol; somatostatin; sparfloxacin; spectinomycin; stavudine; streptokinase; streptozocin; suxamethonium chloride; tacrine hydrochloride; terbutaline sulfate; thiopeta; ticarcillin; tiludronate; timolol; tissue type plasminogen activator; TNFR:Fc; TNK-tPA; trandolapril; trimetrexate gluconate; trospectinomycin; trovafloxacin; tubocurarine chloride; tumor necrosis factor; typhoid vaccine live; urea; urokinase; vancomycin; valacyclovir; valsartan; varicella virus vaccine live; vasopressin and vasopressin derivatives; vecoronium bromide; vinblastine; vincristine; vinorelbine; vitamin B12; warfarin sodium; yellow fever vaccine; zalcitabine; zanamivir; zolendronate; zidovudine; pharmaceutically acceptable salts, isomers and derivatives thereof; and mixtures thereof.

The active ingredient can also be administered in combination with one or more additional active ingredients. Any of the aforementioned active agents may also be administered in combination using the present formulations. Active agents administered in combination may be from the same therapeutic class (e.g., lipid-regulating agents or anticoagulants) or from different therapeutic classes (e.g., a lipid-regulating agent and an anticoagulant). Examples of particularly important drug combination products include, but are not limited to:

female contraceptive compositions containing both a progestogen and an estrogen;

female HRT compositions containing a progestogen, an estrogen, and an androgen;

combinations of lipid-regulating agents, e.g., (a) a fibrate and a statin, such as fenofibrate and atorvastatin, fenofibrate and simvastatin, fenofibrate and lovastatin, or fenofibrate and pravastatin; (b) a fibrate and nicotinic acid, such fenofibrate and niacin; and (c) a statin and a nicotinic acid, such as lovastatin and niacin;

combinations of a lipid-regulating agent and an antiviral agent, e.g., a fibrate and a protease inhibitor, such as fenofibrate and ritonavir;

combinations of a lipid-regulating agent and an anticoagulant, e.g., (a) a fibrate and a salicylate, such as fenofibrate and aspirin, (b) a fibrate and another anticoagulant, such as fenofibrate and clopidogrel, (c) a statin and a salicylate, such as simvastatin and aspirin, and (d) a statin and another anticoagulant such as pravastatin and clopidogrel;

combinations of a lipid-regulating agent and an antidiabetic agent, including (a) a fibrate and a insulin sensitizer such as a thiazolidinedione, e.g., fenofibrate and pioglitazone, or fenofibrate and rosiglitazone, (b) a fibrate and an insulin stimulant such as a sulfonylurea, e.g., fenofibrate and glimepiride, or fenofibrate and glipizide, a statin and and insulin sensitizer such as a thiazolidinedione, e.g., lovastatin and pioglitazone, simvastatin and rosiglitazone, pravastatin and pioglitazone, or the like;

combinations of a lipid regulating agent and a cardiovascular drug, e.g., (a) a fibrate and a calcium channel blocker, such as fenofibrate and amlodipine, or fenofibrate and irbesartan, or (b) a statin and a calcium channel blocker, such as fosinopril and pravastatin;

combinations of anticoagulants, e.g., (a) a salicylate and a platelet receptor binding inhibitor, such as aspirin and clopidogrel, (b) a salicylate and a low molecular weight heparin, such as aspirin and dalteparin, and (c) a platelet receptor binding inhibitor and a low molecular weight heparin, such as clopidogrel and enoxaparin;

combinations of antidiabetics, e.g., (a) an insulin sensitizer and an insulin stimulant, such as (i) a thiazolidinedione such as glitazone or pioglitazone and a sulfonylurea such as glimepiride, and (ii) a biguanide such as metformin and a meglitinide such as repaglinide, (b) an insulin sensitizer and an α-glucosidase inhibitor, such as metformin and acarbose, (c) an insulin stimulant and an α-glucosidase inhibitor, such as (i) a sulfonylurea such as glyburide combined with acarbose, (ii) acarbose and a meglitinide such as repaglinide, (iii) miglitol and a sulfonylurea such as glipizide, or (iv) acarbose and a thiazolidinedione such as pioglitazone;

combinations of cardiovascular drugs, such as combinations of ACE inhibitors, e.g., lisinopril and candesartan; a combination of an ACE inhibitor with a diuretic agent such as losartan and hydrochlorothiazide; a combination of a calcium channel blocker and a β-blocker such as nifedipine and atenolol; and a combination of a calcium channel blocker and an ACE inhibitor such as felodipine and ramipril;

combinations of an antihypertensive agent and an antidiabetic agent, such as an ACE inhibitor and a sulfonylurea, e.g., irbesartan and glipizide;

combinations of antihistamines and antiasthmatic agents, e.g., an antihistamine and a leukotriene receptor antagonist such as loratadine and zafirlukast, desloratidine and zafirlukast, and cetirazine and montelukast;

combinations of antiinflammatory agents and analgesics, e.g., a COX-2 inhibitor and a nonsteroidal antiinflammatory agent (NSAID) such as rofecoxib and naproxen, or a COX-2 inhibitor and a salicylate such as celecoxib and aspirin;

combinations of an anti-obesity drug and an antidiabetic agent, e.g., a lipase inhibitor such as orlistat in combination with metformin;

combinations of a lipid-regulating agent and a drug for treating coronary artery disease, e.g., fenofibrate and ezetimibe, or lovastatin and ezetimibe; and

other combinations, such as docetaxel and cisplatin, tirapazamine and cisplatin, metoclopramide and naproxen sodium, an opioid analgesic such as oxycodone and an anti-inflammatory agent, an agent for treating erectile dysfunction, such as alprostadil, with an antihypertensive/vasodilator such as prazosin.

4. Concentrations

The components of the pharmaceutical compositions of the present invention in amounts such that upon dilution with an aqueous solution, the composition forms a clear, aqueous dispersion. The determining concentrations of components to form clear aqueous dispersions are the concentrations of triglyceride and surfactants, with the amount of the therapeutic agent, if present, being chosen as described below. The relative amounts of triglycerides and surfactants are readily determined by observing the properties of the resultant dispersion; i.e., when the relative amounts of these components are within a suitable range, the resultant aqueous dispersion is optically clear. When the relative amounts are outside the suitable range, the resulting dispersion is visibly “cloudy”, resembling a conventional emulsion or multiple-phase system. Although a visibly cloudy solution may be potentially useful for some applications, such a system would suffer from many of the same disadvantages as conventional prior art formulations, as described above.

A convenient method of determining the appropriate relative concentrations for any particular triglyceride is as follows. A convenient working amount of a hydrophilic surfactant is provided, and a known amount of the triglyceride is added. The mixture is stirred, with the aid of gentle heating if desired, then is diluted with purified water to prepare an aqueous dispersion. Any dilution amount can be chosen, but convenient dilutions are those within the range expected in vivo, about a 10 to 250-fold dilution. In the Examples herein, a convenient dilution of 100-fold was chosen. The aqueous dispersion is then assessed qualitatively for optical clarity. The procedure can be repeated with incremental variations in the relative amount of triglyceride added, to determine the maximum relative amount of triglyceride that can be present to form a clear aqueous dispersion with a given hydrophilic surfactant. I.e., when the relative amount of triglyceride is too great, a hazy or cloudy dispersion is formed.

The amount of triglyceride that can be solubilized in a clear aqueous dispersion is increased by repeating the above procedure, but substituting a second hydrophilic surfactant, or a hydrophobic surfactant, for part of the originally-used hydrophilic surfactant, thus keeping the total surfactant concentration constant. Of course, this procedure is merely exemplary, and the amounts of the components can be chosen using other methods, as desired.

It has been found that mixtures of surfactants including two hydrophilic surfactants can solubilize a greater relative amount of triglyceride than a single surfactant. Similarly, mixtures of surfactants including a hydrophilic surfactant and a hydrophobic surfactant can solubilize a greater relative amount of triglyceride than either surfactant by itself. It is notable that when the surfactant mixture includes a hydrophilic surfactant and a hydrophobic surfactant, the solubility of the triglyceride is greater than, for example, in the hydrophilic surfactant itself. Thus, contrary to conventional knowledge in the art, the total amount of water-insoluble component (triglyceride plus hydrophobic surfactant) exceeds the amount of hydrophobic surfactant that can be solubilized by the same amount of hydrophilic surfactant.

It should be emphasized that the optical clarity is determined in the diluted composition (the aqueous dispersion), and not in the pre-concentrate. Thus, for example, U.S. Pat. No. 4,719,239 shows optically clear compositions containing water, oil, and a 3:7 mixture of PEG-glycerol monooleate and caprylic-capric acid glycerol esters, but the compositions contain no more that about 75% by weight water, or a dilution of the pre-concentrate of no more than 3 to 1. Upon dilution with water in a ratio of more than about 3 to 1, the compositions of the cited reference phase-separate into multi-phase systems, as is shown, for example, in the phase diagram of FIG. 2 in the '239 patent. In contrast, the compositions of the present invention, when diluted to values typical of dilutions encountered in vivo, or when diluted in vivo upon administration to a patient, remain as clear aqueous dispersions. Thus, the clear aqueous dispersions of the present invention are formed upon dilution of about 10 to about 250-fold or more.

As an alternative to qualitative visual assessment of optical clarity, the optical clarity of the aqueous dispersion can be measured using standard quantitative techniques for turbidity assessment. One convenient procedure to measure turbidity is to measure the amount of light of a given wavelength transmitted by the solution, using, for example, a UV-visible spectrophotometer. Using this measure, optical clarity corresponds to high transmittance, since cloudier solutions will scatter more of the incident radiation, resulting in lower transmittance measurements. If this procedure is used, care should be taken to insure that the composition itself does not absorb light of the chosen wavelength, as any true absorbance necessarily reduces the amount of transmitted light and falsely increases the quantitative turbidity value. In the absence of chromophores at the chosen wavelength, suitable dispersions at a dilution of 100× should have an apparent absorbance of less thar about 0.3, preferably less than about 0.2, and more preferably less than about 0.1.

Other methods of characterizing optical clarity, such as direct particle size measurement and other methods known in the art may also be used. It should be emphasized that any or all of the available methods may be used to ensure that the resulting aqueous dispersions possess the requisite optical clarity. For convenience, however, the present inventors prefer to use the simple qualitative procedure; i.e., simple visible observation.

If present, the therapeutic agent is solubilized in the triglyceride, the carrier, or in both the triglyceride and the carrier. Alternatively, the therapeutic agent can be solubilized in the aqueous medium used to dilute the preconcentrate to form an aqueous dispersion. The maximum amount of therapeutic agent that can be solubilized is readily determined by simple mixing, as the presence of any non-solubilized therapeutic agent is apparent upon visual examination.

In one embodiment, the therapeutic agent is present in an amount up to the maximum amount that can be solubilized in the carrier. In another embodiment, the therapeutic agent is present in a first amount which is solubilized, and a second amount that remains unsolubilized but dispersed. This may be desirable when, for example, a larger dose of the therapeutic agent is desired. Of course, in this embodiment, the optical clarity of the resultant aqueous dispersion is determined before the second non-solubilized amount of the therapeutic agent is added. In yet another another embodiment, the therapeutic agent may be suspended in the carrier only, and is present in an amount up to the maximum amount that can be suspended in the carrier while still allowing the dosage form to function in a desired manner.

5. Solubilizers

If desired, the pharmaceutical compositions of the present invention can optionally include additional compounds to enhance the solubility of the therapeutic agent or the triglyceride in the composition. Examples of such compounds, referred to as “solubilizers”, include: alcohols and polyols, such as ethanol, isopropanol, butanol, benzyl alcohol, ethylene, glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives;ethers of polyethylene zlycols having an average molecular weight of about 200 to about 6000, such as tetrahydrofurfuryl alcohol PEG ether (glycofurol, available commercially from BASF under the trade name Tetraglycol) or methoxy PEG (Union Carbide); amides, such as 2-pyrrolidone, 2-piperidone, 6-caprolactam, N-alkylpyrrolidone, N-hydroxyalkylpyrrolidone, N-alkylpiperidone, N-alkylcaprolactam, dimethylacetamide, and polyvinylpyrrolidone; esters, such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, Φ-caprolactone and isomers thereof, Δ-valerolactone and isomers thereof, β-butyrolactone and isomers thereof;and other solubilizers known in the art, such as dimethyl acetamide, dimethyl isosorbide (Arlasolve DMI (ICI)), N-methyl pyrrolidones (Pharmasolve (ISP)), monooctanoin, diethylene glycol monoethyl ether (available from Gattefosse under the trade name Transcutol), and water.

Mixtures of solubilizers are also within the scope of the invention. Except as indicated, these compounds are readily available from standard commercial sources.

Preferred solubilizers include triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-600, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included in fill material of the present invention is not particularly limited. Of course, when such compositions are ultimately administered to a patient, the amount of a given solubilizer is limited to a bioacceptable amount, which is readily determined by one of skill in the art. In some circumstances, it may be advantageous to include amounts of solubilizers far in excess of bioacceptable amounts, for example, to maximize the concentration of therapeutic agent, with excess solubilizer being removed prior to providing the composition to a patient using conventional techniques, such as distillation or evaporation. Thus, if present, the solubilizer can be in a concentration of 50%, 100%, 200%, or up to about 400% by weight, based on the amount of surfactant. If desired, very small amounts of solubilizers may also be used, such as 25%, 10%, 5%, 1% or even less. Typically, the solubilizer will be present in an amount of about 1% to about 100%, more typically about 5% to about 25% by weight or about 10% to about 25% by weight.

6. Stabilizing Agents

The formulations of the present invention optionally include one or more stabilizing agents to increase the stability and/or compatibility of the suspension when formulated into a dosage form. Suitable stabilizing agents are suspending agents, flocculating agents, thickening agents, gelling agents, buffering agents, antioxidants, preservatives, antimicrobial agents, and mixtures thereof. Ideally, the agent acts to minimize irreversible aggregation of suspended particles, and to maintain proper flow characteristics to ease manufacturing processes, e.g., to ensure that the formulation can be readily pumped and filled into desired dosage forms, such as capsules. In some instances, however, it may be desirable that the formulation have a high viscosity, so that no leakage will occur before a capsule or other dosage form is permanently sealed.

A preferred stabilizing agent in most cases is a suspending agent that imparts increased viscosity and retards sedimentation, to prevent caking. A wide variety of pharmaceutically acceptable excipient with such attributes, of the many well known in the art, can be used as such a suspending agent. Suitable suspending agents include cellulose derivatives, clays, natural gums, synthetic gums, or other agents known in the art. Specific suspending agents, by way of example, include without limitation, microcrystalline cellulose, sodium carboxymethylcellulose, powdered cellulose, ethymethylcellulose, hydroyxypropyl methylcellulose, methylcellulose, ethylcellulose, ethylhydroxy ethylcellulose, hydroxypropyl cellulose, attapulgite, bentonite, hectorite, montmorillonite, silica gel, fumed silicon dioxide, colloidal silicon dioxide, acacia, agar, carrageenan, guar gum, locust bean gum, pectin, sodium alginate, propylene glycol alginate, tamarind gum, xanthan gum, carbomer, povidone, sodium starch glycolate, starches, tragacanth, magnesium aluminum silicate, aluminum silicate, magnesium silicate, gelatin, and glycyrrhizin. These suspending agents can further impart different flow properties to the suspension. The flow properties of the suspension can be Newtonian, plastic, pseudoplastic, thixotropic or combinations thereof. Mixtures of suspending agents may also be used to optimize flow properties and viscosity.

The stabilizing agent may also be a flocculating agent that enables particles to associate in loose aggregates or “flocs.” Although these flocs may settle rapidly, they are easily redispersed. Many flocculating agents known in the art can be utilized, including surfactants, hydrophilic polymers, clays, and electrolytes. Any other pharmaceutically acceptable exicipient with such attributes can also be utilized as a flocclulating agent. In some cases, the flocculating agent may serve a dual purpose, serving not only as a stabilizing agent but also, for example, as a component of the solid particles or as a suspending agent. Suitable flocculating agents include, but are not limited to, sodium lauryl sulfate, sodium docusate, benzalkonium chloride, polysorbate 80, sorbitan monolaurate, sodium carboxymethylcellulose, xanthan gum, tragacanth, methylcellulose, magnesium aluminum silicate, attapulgite, bentonite, potassium dihydrogen phosphate, aluminum chloride, and sodium chloride. The formulation may include both a flocculating agent and a suspending agent, so that the sendimentation of flocs can be retarded.

The stabilizing agent may also be a thickening agent, selected to increase the viscosity of the suspension to a degree sufficient to reduce and retard sedimentation of suspended active agent particles. Any pharmaceutically acceptable excipient with such attributes can be used in the present invention. Typically, compounds that soften slightly above ambient temperature are desirable for this purpose. Preferred thickening agents have a melting point greater than about 25° C., and can be reversibly liquified and solidified. With an appropriate amount of such a thickening agent, the formulation as a whole can acquire this thermosoftening property.

7. Other Additives

Other additives conventionally used in pharmaceutical compositions can be included, and these additives are well known in the art. Such additives include detackifiers, anti-foaming agents, buffering agents, antioxidants, preservatives, chelating agents, viscomodulators, tonicifiers, flavorants, colorants odorants, opacifiers, binders, fillers, plasticizers, lubricants, and mixtures thereof. The amounts of such additives can be readily determined by one skilled in the art, according to the particular properties desired.

8. Dosage Forms

As will be evident from a reading of this patent application, the present invention encompasses a variety of specific dosage forms that include the basic elements as recited herein of a highly hydrophilic fill material and a shell encapsulating the fill material. One general category of such dosage form specifically contemplated to be within the scope of the present invention is capsules.

A wide variety of capsules, including methods and materials for the making thereof, are known to those of ordinary skill in the art, such as hard and soft capsules that are either single piece or two piece capsules. Many typical capsules of this nature provide an instant release of the active agent and thus release substantially all of the active agent in a relatively short time period. However, additional steps may be taken to prolong or extend release of the active agent, for example, by adding a coating to the capsule to provide a sustained release formulation. A variety of such coatings are known to those of ordinary skill in the art, such as enteric and osmotic coatings, as well as a number of other mechanisms for prolonging or otherwise altering release of the active agent from the capsule in a desired manner.

Additionally, when two piece capsules are used, a number of techniques are known for banding or sealing the pieces of the capsule together to prevent leakage of the encapsulated fill material. Such processes and techniques may be used in connection with the dosage forms of the present invention, when such dosage forms involve a two piece capsule.

Accordingly, in one aspect, the dosage form of the present invention may be a capsule. In another aspect, the capsule may be a gelatin capsule. In yet another aspect, the gelatin capsule may be a soft gelatin capsule. In a further aspect, the capsule may be a single piece capsule. In an additional aspect, the capsule may be a two piece capsule which is banded or sealed in order to prevent leakage of the encapsulated fill material. In another aspect, the capsule may be an instant release formulation. In a further aspect, the capsule may include one or more mechanisms for varying or sustaining the release of the active agent.

The following examples of oral dosage forms are provided to promote a more clear understanding of the possible combinations of the present invention, and are in no way meant as a limitation thereon.

### EXAMPLES

Compositions of highly hydrophilic fill materials containing high levels of hydrophilic surfactant as used in the the present invention, were formulated into capsule dosage forms employing a traditional soft gelatin shell, known as an airfill, that is suitable for use with moderately hydrophilic fill material such as PEG base-formulations.

### Example 1

| | | |
|---|---|---|
| a) | Fill composition | (% by weight) |

| | |
|---|---|
| Fenofibrate | 8 |
| Cremphor EL | 35 |
| Labrasol | 22 |
| Labrafil M2125CS | 35 |

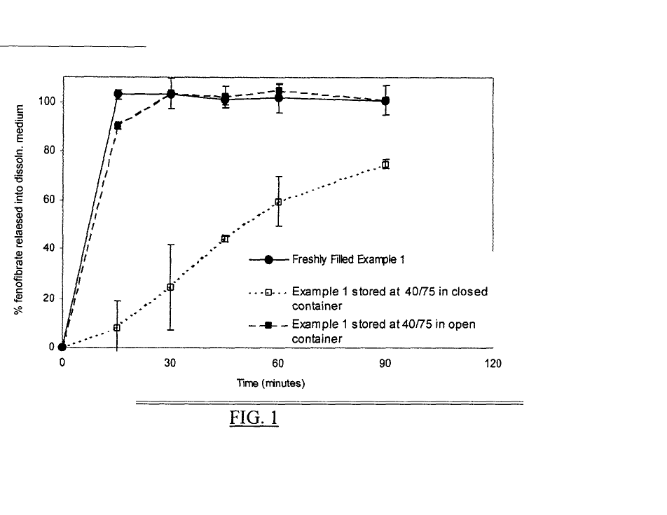

The traditional airfills encapsulating the above-recited fill composition were stored at 40° C./75% RH in closed and an open containers for 4 weeks. These capsules along with freshly filled capsules were subject to dissolution testing (USP type I) in 1 L of SGF with 25 mM sodium lauryl sulfate at 37° C. The release profiles of fenofibrate from the capsules under different storage conditions are demonstrated in FIG. 1

As can be seen, the capsules stored at 40° C./75% RH in a closed container produced a slower and incomplete release of fenofibrate. It also should be noted that there were ghost capsules or the pellicle formation observed from the capsules. These observations highlight the incompatibility between the highly hydrophilic fill material containing more than 40% by weight of hydrophilic surfactants in the carrier and the traditional gelatin capsule shell designed for a PEG-based hydrohpilic formulation.

Notably, when the same capsules were stored in an open container and thus directly exposed to the high humidity, the disintegration/dissolution of the capsules and the release of fenofibrate from the capsules comparable to capsules of the same composition that were freshly filled. Without wishing to bound by and theory, it is thought that the moisture from the humid environment was absorbed by the gelatin shell and consequently provided sufficient plasticity to the shell to compensate for the lose of the fill soluble plasticizer or water migrating into the highly hydrophilic fill material. It is also thought that the plasticizing effect from the absorbed moisture might reduce or inhibit any potential physical or chemical change occurred to the capsules, such as physical denaturing of the gelatin caused by dehydration, deplasticizing or collapsing of the gelatin matrix structure or chemical crosslinking of the gelatin such that the capsule shell was compatible with the highly hydrophilic fill material of the present invention. It is therefore concluded that by providing an extra amount of the plasticizer and/or partially or completely replacing the migratible plasticizer or water with a plasticizing agent of lower solubility in the fill material thus lower tendency of migrating into the fill material, the compatibility can be improved.

Various examples of gelatin capsules in accordance with the present invention having are recited below:

### Example 2

| a) | Fill composition | (% by weight) |
|---|---|---|
| | Fenofibrate | 8 |
| | Cremphor EL | 42 |
| | Labrasol | 20 |
| | Labrafil M2125CS | 30 |
| b) | Shell (dry) composition | (% by weight) |
| | Gelatin | 54 |
| | Glycerin | 18 |
| | Sorbitol/Sorbitan(s) mixture | 22 |
| | Water | 6 |

The dry gelatin shell (capsule) is produced from a fluid gelatin
composition using the following constituents:

| c) | Shell (fluid formation) | (% by weight) |
|---|---|---|
| | Gelatin | 42 |
| | Glycerol | 10 |
| | Sorbitol/Sorbitan(s) mixture | 12 (R 2*) |
| | Water | 36 |

### Example 3

| a) | Fill composition | (% by weight) |
|---|---|---|
| | Fenofibrate | 12 (4 parts suspended) |
| | Cremphor EL | 40 |
| | Labrasol | 26 |
| | Labrafil M2125CS | 22 |
| | Lutrol F68 | 2 |
| b) | Shell (dry) composition | (% by weight) |
| | Gelatin | 47 |
| | Glycerin | 28 |
| | Sorbitol/Sorbitan(s) mixture | 15 (R 1*) |
| | Water | 10 |

The sorbitol/sorbitan(s) mixtures usually contain sorbitol and at least one
sorbitan in a ratio (R)* of sorbitol to sorbitan(s) ranging from about 0.2 to
5, preferably from about 0.5 to 2.5 by weight. Sorbitol/sorbitan(s) mix-
tures are commercially available under various trade names. Due to their
particular processes, these mixtures of sorbitol and sorbitan(s) may further
include other polyhydric alcohol(s) such as mannitol, isosorbide or other
polyols. In these cases, the total amount of sorbitol and sorbitan(s) in the
whole sorbitol/sorbitan(s)/polyhydric alcohol(s) mixtures ranges from
40-80% by weight, preferably from 50-70% by weight. One suitable
commercially available mixture of sorbitol/sorbitans/polyhydric alcohols
is under the trade name “Anidrisorb”. This typical composition of this
product is as follows:

| c) | Constituent | (% by weight) |
|---|---|---|
| | D-Sorbitol | 20-50 |
| | Sorbitans | 20-30 |
| | Mannitol | 0-10 |
| | Other polyols (hydrogenated saccharides) | 20-25 |
| | Water | 10-20 |

### Example 4

| a) | Fill composition | (% by weight) |
|---|---|---|
| | Progesterone | 5 |
| | TPGS | 67 |
| | Cremophor EL | 6 |
| | Labrafil M2125CS | 3 |
| | Ethanol | 14 |
| | Propylene Glycol | 5 |
| b) | Shell (dry) composition | (% by weight) |
| | Gelatin | 51 |
| | Glycerin | 32 |
| | Anidrisorb 35/70 | 12 |
| | Water | 5 |

The dry gelatin shell (capsule) is produced from a fluid gelatin
composition using the following constituents:

| c) | Shell (fluid formation) | (% by weight) |
|---|---|---|
| | Gelatin | 38 |
| | Glycerin | 21 |
| | Anidrisorb 35/70 | 9 |
| | Water | 32 |

### Example 5

| a) | Fill composition | (% by weight) |
|---|---|---|
| | Progesterone | 12 (8 parts suspended) |
| | TPGS | 28 |
| | Cremophor EL | 22 |
| | Labrafil M2125CS | 18 |
| | α-tocopherol | 12 |
| | Propylene Glycol | 8 |
| b) | Shell (dry) composition | (% by weight) |
| | Gelatin | 51 |
| | Glycerin | 32 |
| | Anidrisorb 35/70 | 12 |
| | Water | 5 |

### Example 6

| a) | Fill composition | (% by weight) |
|---|---|---|
| | Progesterone | 4 |
| | Cremophor EL | 42 |
| | Maisine 1-35 | 27 |
| | Miglyol 812 | 26 |
| b) | Shell (dry) composition | (% by weight) |
| | Gelatin | 53 |
| | Glycerin | 35 |
| | Water | 12 |

The dry gelatin shell (capsule) is produced from a fluid gelatin
composition using the following constituents:

| c) | Shell (fluid formation) | (% by weight) |
|---|---|---|
| | Gelatin | 38 |
| | Glycerin | 26 |
| | Water | 36 |

### Example 7

| a) | Fill composition | (% by weight) |
|---|---|---|
| | Progesterone | 4 |
| | Cremophor EL | 42 |
| | Maisine 1-35 | 27 |
| | Miglyol 812 | 26 |
| b) | Shell composition for encapsulation (Wet) | (% by weight) |
| | Gelatin | 48 |
| | Glycerin | 10 |
| | Propylene Glycol | 6 |
| | Triacetin | 4 |
| | Water | 32 |

### Example 8

| a) | Fill composition | (% by weight) |
|---|---|---|
| | Progesterone | 12 (8 parts suspended) |
| | Estradiol | 0.1 |
| | TPGS | 21 |
| | Cremophor EL | 24 |
| | Labrafil M2125CS | 20 |
| | α-tocopherol | 11 |
| | Propylene Glycol | 8 |
| | Water | 4 |
| b) | Shell (dry) composition | (% by weight) |
| | Gelatin | 50 |
| | Starch | 15 |
| | Maltitol Syrup | 25 |
| | Water | 10 |

### Example 9

| a) | Fill composition | (% by weight) |
|---|---|---|
| | Ondansetron | 2 |
| | Cremophor RH40 | 40 |
| | Capmul MCM | 20 |
| | PEG 400 | 20 |
| | Propylene Glycol | 8 |
| | Ethanol | 10 |
| b) | Shell composition for encapsulation (Wet) | (% by weight) |
| | Gelatin | 35 |
| | Gum Acacia | 9 |
| | Glycerin | 7 |
| | Sorbitol solution | 18 |
| | Maltitol solution | 6 |
| | Water | 25 |

### Example 10

| a) | Fill composition | (% by weight) |
|---|---|---|
| | Sumatriptan | 10 (9 parts suspended) |
| | Cremophor RH40 | 40 |
| | Capmul MCM | 28 |
| | PEG 400 | 28 |
| b) | Shell composition for encapsulation (Wet) | (% by weight) |
| | Gelatin | 42 |
| | Sorbitol solution | 30 |
| | Water | 28 |

### Example 11

| a) | Fill composition | (% by weight) |
|---|---|---|
| | Zolpidem | 2 (1.5 parts suspended) |
| | Cremophor RH40 | 65 |
| | PVP K-12 | 30 |
| | Water | 3 |
| b) | Shell composition for encapsulation (Wet) | (% by weight) |
| | Gelatin | 47 |
| | Sorbitol solution | 6 |
| | PEG 200 | 15 |
| | Water | 32 |

### Example 12

| | | (% by weight) |
|---|---|---|
| a) | Fill composition | |
| | Zaleplon | 2 (1.5 parts suspended) |
| | TPGS | 70 |
| | Propylene Glycol | 8 |
| | Ethanol | 15 |
| | Water | 5 |
| b) | Shell composition for encapsulation (Wet) | |
| | Gelatin | 47 |
| | Glycerin | 10 |
| | Sorbitol solution | 6 |
| | Acetylatd monoglyceride | 5 |
| | Water | 32 |

It is to be understood that the above-described arrangements are only illustrative of the application of the principles of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that numerous modifications, including, but not limited to, variations in size, materials, shape, form, function and manner of operation, assembly and use may be made without departing from the principles and concepts set forth herein.

What is claimed is:

1. A pharmaceutical dosage form comprising:
a fill material including a carrier of at least about 40% w/w of a hydrophilic surfactant, and at least one pharmaceutically active agent; and
a shell encapsulating the fill material, said shell containing at least one plasticizing agent in an amount sufficient to maintain an effective shell plasticity upon migration of a portion of the plasticizing agent into the fill material.

2. A pharmaceutical dosage form comprising:
a fill material including a carrier of at least about 40% w/w of a hydrophilic surfactant, and at least one pharmaceutically active agent; and
a shell encapsulating the fill material, said shell containing an effective amount of a plasticizing agent having a solubility of less than 10% w/w in the fill material.

3. The pharmaceutical dosage form of either claim 1 or 2, further comprising a lipophilic coating on an outer surface of the shell.

4. The pharmaceutical dosage form of either claim 1 or 2, wherein the hydrophilic surfactant is a member selected from the group consisting essentially of: non-ionic surfactants having an HLB value of equal to or greater than 10, ionic surfactants, and mixtures thereof.

5. The pharmaceutical dosage form of claim 4, wherein the hydrophilic surfactant is a non-ionic surfactant having an HLB value of equal to or greater than 10.

6. The pharmaceutical dosage form of either claim 1 or 2, wherein the carrier further comprises a lipophilic additive selected from the group consisting essentially of: lipophilic surfactants, triglycerides, and mixtures thereof.

7. The pharmaceutical dosage form of claim 6, wherein the lipophilic additive is a lipophilic surfactant.

8. The pharmaceutical dosage form of claim 7, wherein the lipophilic surfactant is a member selected from the group consisting essentially of: non-ionic surfactants having an HLB value of equal to or less than 10, unionized ionizable surfactants, and mixtures thereof.

9. The pharmaceutical dosage form of claim 8, wherein the lipophilic surfactant is a non-ionic surfactant having an HLB value of equal to or less than 10.

10. The pharmaceutical dosage form of claim 7, wherein the hydrophilic surfactant and the lipophilic surfactant are presented in the carrier at respective amounts such that upon diluting with an aqueous solution, the carrier forms a clear aqueous dispersion.

11. The pharmaceutical dosage form of claim 10, wherein the surfactants are diluted with an aqueous solution at an aqueous solution to carrier ratio of about 100:1 by weight, and the clear aqueous dispersion has an absorbance of less than about 0.3 at 400 nm.

12. The pharmaceutical dosage form of either claim 1 or 2, wherein the at least one pharmaceutically active agent is a lipophilic drug.

13. The pharmaceutical dosage form of either claim 1 or 2, wherein the at least one pharmaceutically active agent is a hydrophilic drug.

14. The pharmaceutical dosage form of either claim 1 or 2, wherein the carrier further comprises at least one additive selected from the group consisting of: antioxidants, bufferants, antifoaming agents, detackifiers, preservatives, chelating agents, viscomodulators, tonicifiers, flavorants, colorants, odorants, opacifiers, stabilizing agents, solubilizers, binders, fillers, plasticizing agents, lubricants, and mixtures thereof.

15. The pharmaceutical dosage form of claim 14, wherein the at least one additive is a solubilizer.

16. The pharmaceutical dosage form of claim 15, wherein the solubilizer is a member selected from the group consisting of alcohols, polyols, amides, esters, propylene glycol ethers, and mixtures thereof.

17. The pharmaceutical dosage form of either claim 1 or 2, wherein the pharmaceutically active agent is at least partially suspended in the carrier.

18. The pharmaceutical dosage form of claim 17, wherein the carrier further includes a stabilizing agent.

19. The pharmaceutical dosage form of claim 18, wherein the stabilizing agent imparts increased viscosity to the fill material and retards sedimentation.

20. The pharmaceutical dosage form of claim 19, wherein the stabilizing agent is a member selected from the group consisting essentially of: suspending agents, flocculating agents, thickening agents, gelling agents, buffering agents, antioxidants, preservatives, antimicrobial agents, and mixtures thereof.

21. The pharmaceutical dosage form of claim 19, wherein the stabilizing agent is a suspending agent.

22. The pharmaceutical dosage form of claim 20, wherein the suspending agent is a member selected from the group consisting essentially of: microcrystalline cellulose, sodium carboxymethylcellulose, powdered cellulose, ethymethylcellulose, hydroyxypropyl methylcellulose, methylcellulose, ethylcellulose, ethylhydroxy ethylcellulose, hydroxypropyl cellulose, attapulgite, bentonite, hectorite, montmorillonite, silica gel, fumed silicon dioxide, colloidal silicon dioxide, acacia, agar, carrageenan, guar gum, locust bean gum, pectin, sodium alginate, propylene glycol alginate, tamarind gum, xanthan gum, carbomer, povidone, sodium starch glycolate, starches, tragacanth, magnesium aluminum silicate, aluminum silicate, magnesium silicate, gelatin, and glycyrrhizin, and mixtures thereof.

23. The pharmaceutical dosage form of either claim 1 or 2, wherein the shell further comprises at least one matrix forming material selected from the group consisting of: gelatin, starch, gum acacia, hydroxypropyl methyl cellulose, and mixtures thereof.

24. The pharmaceutical dosage form of claim 23, wherein the at least one matrix forming material is gelatin.

25. The pharmaceutical dosage form of claim 1, wherein the solubility of the plasticizing agent in the fill material is less than about 10% w/w.

26. The pharmaceutical dosage form of either claim 1 or 25, wherein the solubility of the plasticizing agent in the fill material is less than about 5% w/w.

27. The pharmaceutical dosage form of either claim 1 or 25, wherein the solubility of the plasticizing agent in the fill material is less than about 1% w/w.

28. The pharmaceutical dosage form of either claim 1 or 25, wherein the solubility of the plasticizing agent in the fill material is less than about 0.5% w/w.

29. The pharmaceutical dosage form of either claim 1 or 2, wherein the amount of plasticizing agent in the shell is from about 4% w/w to about 60% w/w of the shell.

30. The pharmaceutical dosage form of either claim 2 or 25, wherein the plasticizing agent is a member selected from the group consisting essentially of: sorbitol, sorbitanes, xylitol, maltitol, maltitol syrup, partially dehydrated hydrogenated glucose syrups, hydrogenated starch hydrolysate, polyhydric alcohols having an equilibrium relative humidity of greater than or equal to 80%, carrageenan, polyglycerol, non-crystallizing solutions of sorbitol, glucose, fructose, glucose syrups, and mixtures and equivalents thereof.

31. The pharmaceutical dosage form of claim 30, wherein the plasticizing agent is a member selected from the group consisting of sorbitol, sorbitanes, maltitol, mannitol, and mixtures thereof.

32. The pharmaceutical dosage form of either claim 1 or 2, wherein the shell further comprises an additive selected from the group consisting of: coloring agents, antioxidant, preservatives, surfactants, and mixtures thereof.

33. The pharmaceutical dosage form of claim 32, wherein the shell additive is a surfactant.

34. The pharmaceutical dosage form of either claim 1 or 2, wherein the dosage form is a capsule.

35. The pharmaceutical dosage form of claim 34, wherein the capsule is a hard capsule.

36. The pharmaceutical dosage form of claim 34, wherein the capsule is a soft capsule.

37. A pharmaceutical dosage form comprising:
a fill material including a carrier of at least about 40% w/w of a hydrophilic surfactant, and at least one pharmaceutically active agent; and
a shell encapsulating the fill material, said shell containing a first plasticizing agent and a second plasticizing agent, said second plasticizing agent having a solubility in the fill material of less than about 10% w/w agent, wherein the first and second plasticizing agents are present in amounts sufficient to maintain an effective shell plasticity upon migration of a portion of either plasticizing agent into the fill material.
